(19) United States
(12) Patent Application Publication
(10) Pub. No.: US 2018/0000324 A1
(43) Pub. Date: Jan. 4, 2018

(54) SHEATH FOR HAND-HELD AND ROBOTIC LAPAROSCOPES

(71) Applicants: Wayne L. POLL, New Albany, OH (US); Gregory P. DRACH, Liberty Township, OH (US); Jeffrey W. STONE, Lebanon, OH (US); Jerome E. RECKELHOFF, Cincinnati, OH (US)

(72) Inventors: Wayne L. POLL, New Albany, OH (US); Gregory P. DRACH, Liberty Township, OH (US); Jeffrey W. STONE, Lebanon, OH (US); Jerome E. RECKELHOFF, Cincinnati, OH (US)

(21) Appl. No.: 15/703,402

(22) Filed: Sep. 13, 2017

Related U.S. Application Data
(63) Continuation of application No. 14/308,644, filed on Jun. 18, 2014.
(60) Provisional application No. 61/836,643, filed on Jun. 18, 2013.
(60) Provisional application No. 61/927,411, filed on Jan. 14, 2014.

(51) Int. Cl. A61B 1/00; A61B 1/015; A61B 34/30; A61B 1/313; A61B 1/12

(52) U.S. Cl. CPC A61B 1/00135; A61B 1/313; A61B 1/126; A61B 34/30; A61B 1/00091; A61B 1/00089; A61B 1/015

(57) ABSTRACT

A sheath for providing a dynamic air shield relative to a distal portion of a laparoscope disposed within the sheath includes a sheath elongate body having an inner surface and an outer surface, a plurality of lumens, and a distal portion configured to deflect air from the lumens across the surface of a laparoscope. The sheath can include a registration and alignment feature configured to align the sheath with a laparoscope. Further, the sheath can be configured to work with robotic laparoscope systems.

### CROSS REFERENCE TO RELATED APPLICATIONS

This application is a continuation of U.S. patent application Ser. No. 14/308,644, filed Jun. 18, 2014, titled “SHEATH FOR HAND-HELD AND ROBOTIC LAPAROSCOPES”, which claims priority to U.S. Provisional Patent Application No. 61/836,643, filed Jun. 18, 2013, titled “LAPAROSCOPIC SHEATH” and to U.S. Provisional Application No. 61/927,411, filed Jan. 14, 2014, titled “LAPAROSCOPIC SHEATH,” all of which are incorporated by reference in their entireties.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

A number of view optimizing devices and systems have been proposed to solve the challenges associated with intra-operative defogging, surgical debris deflection, and cleaning of a laparoscopic lens during minimally invasive surgery. Examples of such devices and systems are found, for example, in U.S. Patent Application Publication No. 2010/0198014 (“the '8014 application”) entitled “Systems and Methods for Optimizing and Maintaining Visualization of a Surgical Field During the Use of Surgical Scopes” and filed U.S. Patent Application Publication No. 2012/0197084 (“the '7084 application”) entitled “Systems and Methods for Optimizing and Maintaining Visualization of a Surgical Field During the Use of Surgical Scopes,” each of which is incorporated herein by reference in its entirety.

In general, one challenge facing these laparoscopic devices and systems is how to ensure that the scope is stable and properly positioned within the sheath. In many cases, a stable and properly registered fit between the surgical scope and the sheath is critical to the effective operation of the system. Stability refers typically to the minimization, prevention, and/or elimination of rotational movement between the scope and the sheath. Registration is a type of positioning used to provide and maintain the proper relation of the distal-most portion of the sheath with the distal-most portion of the scope. In some designs, one factor for such positioning is to establish and maintain a gap between the distal-most ends of the sheath-scope. In addition, proper positioning may include features to prevent rotation or other relative movement between the scope and the sheath. While some designs have been proposed in the aforementioned published patent applications, challenges remain as a result of physical design constraints alone or in combination with other equipment in the surgical operating field. The need remains for improved designs and techniques for providing stability and registration between a sheath and a surgical device.

Another challenge with the laparoscopic devices and systems is getting the sheath to work with robotic systems and laparoscopes. Robotic surgeries are becoming increasingly common. However, clear visualization is one of the biggest problems facing the field of surgical robotics. Using a defogging sheath to clear visualization could therefore be advantageous for such surgeries. However, there are specific challenges associated with doing so, including attachment of the sheath to the robotic system, automated control, and adjustment of the sheath to work with bifocal lenses.

The systems described herein are geared towards addressing some of these challenges.

### SUMMARY OF THE DISCLOSURE

In general, in one embodiment, a sheath for providing a dynamic air shield relative to a distal portion of a surgical instrument disposed within the sheath includes a sheath elongate body having an inner surface and an outer surface. A distal portion of the sheath elongate body has a deflector extending from the outer surface towards and beyond the inner surface, a plurality of stand-offs on the deflector sized to space a distal most portion of the surgical instrument apart from the deflector, a connector piece, and a resilient member coupled to a proximal portion of the sheath elongate body. A plurality of lumens extends between the inner surface and the outer surface. The inner surface has an interior dimension selected to receive the surgical instrument. The deflector extends at least partially around the perimeter of the sheath elongate body. When the surgical instrument is disposed within the sheath, the connector piece is coupled to the surgical instrument so as to distend the resilient member to maintain apposition between the distal most portion of the surgical instrument and the plurality of stand-offs.

This and other embodiments can include one or more of the following features. A rigid distal portion of the surgical instrument can be positioned adjacent to a distal portion of the sheath elongate body proximate to the deflector. The resilient member can be a spring, a pneumatic actuator, or a rubber band. The resilient member can be configured to provide 8-12 lb of force to maintain the apposition. The resilient member can be configured to provide 1-5 lb of force to maintain the apposition. The connector piece can be adapted and configured for positioning between two existing surgical instrument components or accessories. The existing surgical instrument component can be an accessory for use with an optics system on the surgical instrument. The surgical instrument can be a laparoscope. The surgical instrument can be a robotic laparoscope. The connector can be a flat annular ring. The connector can include a keying feature configured to align the sheath with the surgical instrument. The device can further include a manifold on the sheath elongate body having at least one inlet for gas and an interior portion configured to provide communication between the inlet and the proximal portion of each of the plurality of lumens. The manifold can include a keying mechanism configured to mate with a mating portion of the surgical instrument to prevent rotation of the sheath relative to the surgical instrument. The mating portion of the surgical instrument can be an accessory for use with an optics system on the surgical instrument. The accessory can include an asymmetric cut-out configured to mate with the keying mechanism.

In general, in one embodiment, a sheath for providing a dynamic air shield relative to a distal portion of a robotically controlled surgical instrument disposed within the sheath includes a sheath elongate body having an inner surface and an outer surface and a plurality of lumens extending between the inner surface and the outer surface. A distal portion of the sheath elongate body has a deflector extending from the outer surface towards and beyond the inner surface, a plurality of stand-offs on the deflector sized to space a distal most portion of the surgical instrument apart from the deflector, and a resilient member coupled to a proximal portion of the sheath elongate body. The inner surface has an interior dimension selected to receive the surgical instrument. The deflector extends at least partially around the perimeter of the sheath elongate body. When the robotically controlled surgical instrument is disposed within the sheath, the resilient member moves from a contracted configuration to an extended configuration such that the resilient member maintains an apposition force between the distal most portion of the robotically controlled surgical instrument and the plurality of stand-offs.

This and other embodiments can include one more of the following features. The apposition force can be sufficient to maintain the distal most portion of the robotically controlled surgical instrument in contact with the stand-offs while the robotically controlled surgical instrument is used to perform a portion of a robotic surgery procedure. The apposition force can be sufficient to maintain the distal most portion of the robotically controlled surgical instrument in contact with the stand-offs while the robotically controlled surgical instrument is inserted into or withdrawn from a surgical field used for a robotic surgery procedure. A rigid distal portion of the robotically controlled surgical instrument can be positioned adjacent to a distal portion of the sheath elongate body proximate to the deflector. The resilient member can be a spring, a pneumatic actuator, or a rubber band. The resilient member can be configured to provide 8-12 lb of force to maintain the apposition. The sheath can further include a connector piece adapted and configured for positioning between two existing robotically controlled surgical instrument components or accessories. The existing robotically controlled surgical instrument component can be an accessory for use with an optics system on the robotically controlled surgical instrument. The connector can have a shape adapted and configured for engagement with or between one or more components of the robotically controlled surgical instrument so as to maintain the resilient member in the extended configuration.

In general, in one embodiment, a sheath for providing a dynamic air shield relative to a distal portion of a surgical instrument having two lenses and two light elements that is disposed within the sheath includes a sheath elongate body having an inner surface and an outer surface and a plurality of lumens extending between the inner surface and the outer surface. A distal portion of the sheath elongate body has a deflector extending from the outer surface towards and beyond the inner surface. A plurality of stand-offs on the deflector are sized to space the distal most portion of the surgical instrument apart from the deflector. The inner surface has an interior dimension selected to receive the surgical instrument. The deflector extends at least partially around the perimeter of the sheath elongate body and includes an elongate window therein configured to expose both of the lenses of the surgical instrument.

This and other embodiments can include one or more of the following features. The sheath can include first, second, third, and forth lumens. The first and second lumens can be configured to provide higher airflow velocity therethrough than the third and fourth lumens. The first and second lumens can be adjacent to one another. The first and second lumens can be closer to an air inlet at the proximal end of the sheath than the third and fourth lumens. The outer surface can include a plurality of cut-outs at a proximal end of the sheath such that a plenum of air forms over the proximal ends of the lumens when air is provided to the sheath through an air inlet. The deflector can include a plurality of dividers that extend substantially in line with walls separating the lumens. The dividers can be configured to sit against an outer diameter of the surgical instrument. The deflector can be configured to at least partially overlap with both of the light elements at the distal end. A flow of gas through the lumens and across the lenses can create a vortex over the distal end of the surgical instrument. The tip of the surgical instrument can be angled to include a nose and a heal. The vortex can start at the heal of the instrument. The vortex can form in a lower right-hand corner of a camera screen associated with the surgical instrument. An axis of the vortex can be substantially transverse to a longitudinal axis of the surgical instrument. The plurality of lumens can be configured to allow air to flow therethrough and over a distal end of the surgical instrument. The sheath can further include an additional lumen configured to allow fluid to flow therethrough and over a distal end of the surgical instrument. The opening can have a generally ovoid shape. The surgical instrument can be a laparoscope. The surgical instrument can be a robotically controlled laparoscope. The stand-offs can be positioned so as to not overlap with any of the light elements at the distal end of the surgical instrument.

In general, in one embodiment, a system for providing a dynamic air shield relative to a distal portion of a robotic surgical instrument disposed within the sheath includes a sheath elongate body having an inner surface and an outer surface and a plurality of lumens extending between the inner surface and the outer surface. An attachment mechanism is configured to connect the sheath to the surgical instrument. A gas connection port is configured to provide gas to one or more of the lumens. A fluid connection portion is configured to provide fluid to one or more of the lumens. A deflector assembly attached to a distal end of the sheath is configured to deflect fluid from the lumens over a distal end of the surgical instrument. An activation element is remotely located relative to the sheath and is configured to provide gas or fluid to the lumens when activated such that the gas or fluid flows over a distal end of the surgical instrument. The inner surface has an interior dimension selected to receive the surgical instrument.

This and other embodiments can include one or more of the following features. The activation element can be located on a console. The console can include elements configured to control the robotic surgical instrument. The activation element can be a foot pedal of the console. The activation element can be a hand control of the console. The activation element can be a voice activated element.

In general, in one embodiment, a method of maintaining a visual field provided by a vision instrument during a robotically assisted surgical procedure includes: (1) positioning a robotically manipulated instrument within a surgical field; (2) disposing the vision instrument in relation to the surgical field or the robotically manipulated instrument to provide visual information related to the robotically manipulated instrument; (3) generating surgical debris or smoke by operation of the robotically manipulated instrument; and (4) operating a dynamic air shield adjacent to the distal portion of the vision instrument such that the output of the vision instrument remains substantially free from obstruction from the surgical debris or smoke.

This and other embodiments can include one or more of the following features. The method can further include applying a cleaning solution to the vision instrument and thereafter altering a characteristic of the dynamic air shield. The cleaning solution can be a biocompatible surfactant. The applying step can be performed without removing the vision instrument from the surgical field. Operating a dynamic air shield can include controlling the dynamic air shield from a console remotely located relative to the dynamic air shield. The surgical instrument can be kept in place without removal for cleaning throughout the entire surgical procedure.

In general, in one embodiment, a method of maintaining a visual field provided by a vision instrument during a robotically assisted surgical procedure includes: (1) positioning a robotically manipulated instrument within a surgical field; (2) disposing the vision instrument in relation to the surgical field or the robotically manipulated instrument to provide visual information related to the robotically manipulated instrument or the surgical field; (3) generating surgical debris or smoke by operation of the robotically manipulated instrument that diminishes the quality of the visual information provided by the vision instrument; (4) delivering a biocompatible surfactant to one or more lens of the vision instrument; (5) operating a dynamic air shield adjacent to the one or more lens of the vision instrument in a first operational state during or immediately after the delivering step; and (6) operating the dynamic air shield adjacent to the one or more lens of the vision instrument in a second operational state after operating in the first operational state for a time period.

This and other embodiments can include one or more of the following features. All steps can be performed without removing the vision instrument from the surgical field. The time period can be determined by a user operating the robotically manipulated instrument. The time period can be a pre-set period. The first operational state can be selected to remove the biocompatible surfactant from the one or more lenses of the vision instrument. Gas can flow over the lens at a higher flow velocity in the first operational state than the second operational state. The second operational state can be selected to defog the one or more lenses of the vision instrument. The surgical instrument can be kept in place without removal for cleaning throughout the entire surgical procedure.

In general, in one embodiment, a method of maintaining a visual field provided by a robotically manipulated instrument during a robotically assisted surgical procedure includes: (1) positioning a robotically manipulated instrument within a robotic surgical field; (2) generating surgical debris or smoke within the robotic surgical field; (3) operating a dynamic air shield adjacent to the distal end of the robotically manipulated instrument during the generating step to prevent a portion of the smoke or the surgical debris from impairing a visual signal provided by the robotically manipulated instrument; (4) operating the dynamic air shield in a first operational configuration while the dynamic air shield prevents the smoke or surgical debris from accumulating on or near the distal portion of the robotically manipulated instrument; and (5) operating the dynamic air shield in a second, different operational configuration if surgical debris has impaired a vision system in the distal portion of the robotically manipulated instrument.

This and other embodiments can include one or more of the following features. The dynamic air shield can be configured to operate adjacent to one or more lens positioned in the distal most portion of the robotically manipulated surgical instruments. The first operational condition can include only the operation of the dynamic flow shield. The method can further include operating one or more activation elements within an operating system used for performing the robotically assisted surgery to perform one or more of a shift between the first operational condition and the second operational condition, an altering of a characteristic of the dynamic air flow shield, or a providing of a fluid. Operating the dynamic air shield in a different operation configuration can include delivering a biocompatible surfactant to one or more lenses of the vision instrument. The surgical instrument can be kept in place without removal for cleaning throughout the entire surgical procedure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an exemplary sheath assembly for cleaning and/or defogging a scope.

FIG. 2 shows an angled connection between tubing and a sheath manifold.

FIG. 3 shows the placement of lumens of a sheath relative to features of a deflector assembly. The view is from the proximal end of the sheath looking distally.

FIG. 4 shows the circumferential dimensions of the lumens of the sheath (viewed distally from the proximal end).

FIGS. 5A-5C show placement of the sheath over a binocular lens.

FIG. 6A shows the inner surfaces of a deflector assembly.

FIG. 6B shows the outer surfaces of a deflector assembly.

FIGS. 7A-7B shows the flow of air and fluid across the inner surfaces of a deflector assembly.

FIGS. 8A-8B show cut-outs at the proximal end of a sheath.

FIGS. 9A-9B show cross-sections of the sheath and fluid/air flow therethrough.

FIGS. 10A-10B shows formation of a vortex over a binocular lens.

FIG. 11 shows an exemplary laparoscopic sheath, laparoscope, and camera mount.

FIG. 12 shows the proximal end of the laparoscopic sheath of FIG. 11.

FIGS. 13A and 13B shows placement of a connector piece of a laparoscopic sheath against a proximal portion of the laparoscope.

FIGS. 14-15B show a mechanism for locking a laparoscopic sheath onto a camera mount and laparoscope.

FIGS. 16A-16C show exemplary keying mechanisms for rotationally locking a sheath relative to a laparoscope.

FIG. 17 shows exemplary movement of a resilient feature of a laparoscopic sheath for axially stabilizing a sheath relative to a laparoscope.

FIG. 18 shows an exemplary robotic system for use with a cleaning/defogging sheath.

FIGS. 19 and 20 show connection of a sheath to a robotic system.

FIG. 21 shows exemplary steps for attaching a sheath to a robotic system.

FIG. 22 shows use of the sheath with a robotic system having a robot, console, and tower.

FIG. 23 is a schematic drawing showing use of the sheath with a robotic system.

FIG. 24 is a schematic showing a controller configured to control the sheath when used with a robotic system.

FIGS. 25A-25D show different user-activated control mechanisms on a console of a robotic system.

FIG. 26 is an exemplary flow chart for use of the sheath with a robotic system.

### DETAILED DESCRIPTION

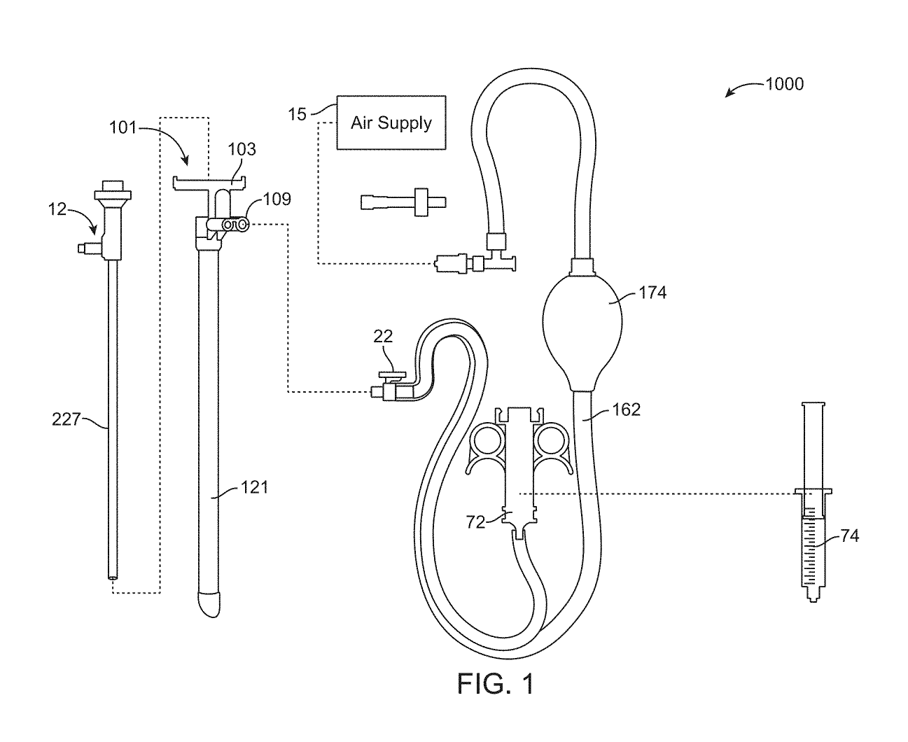

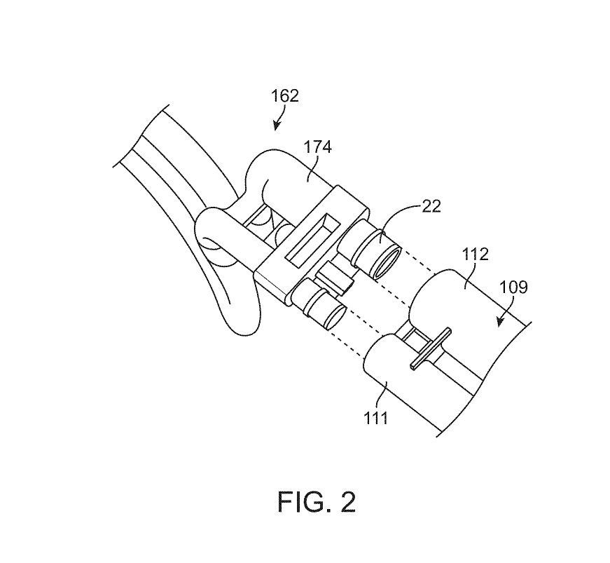

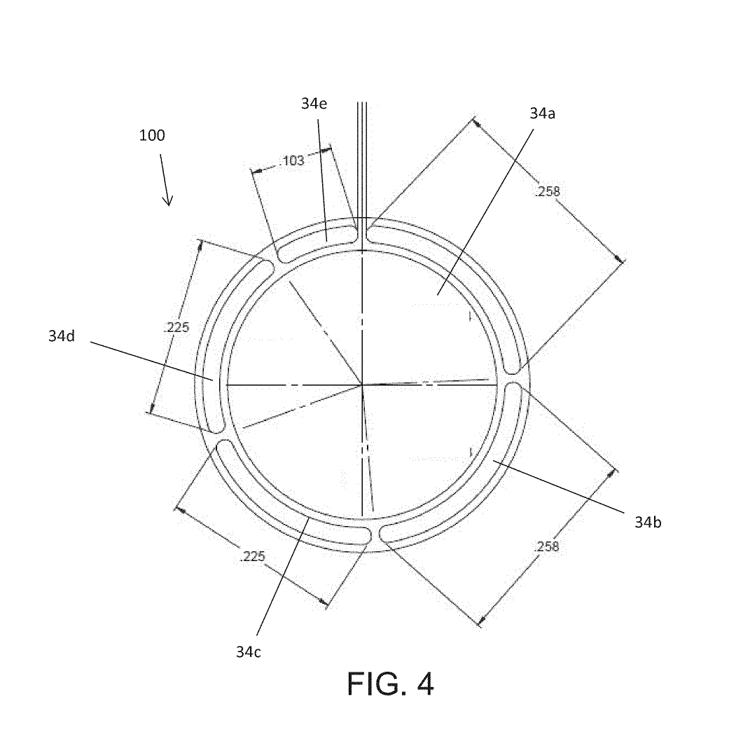

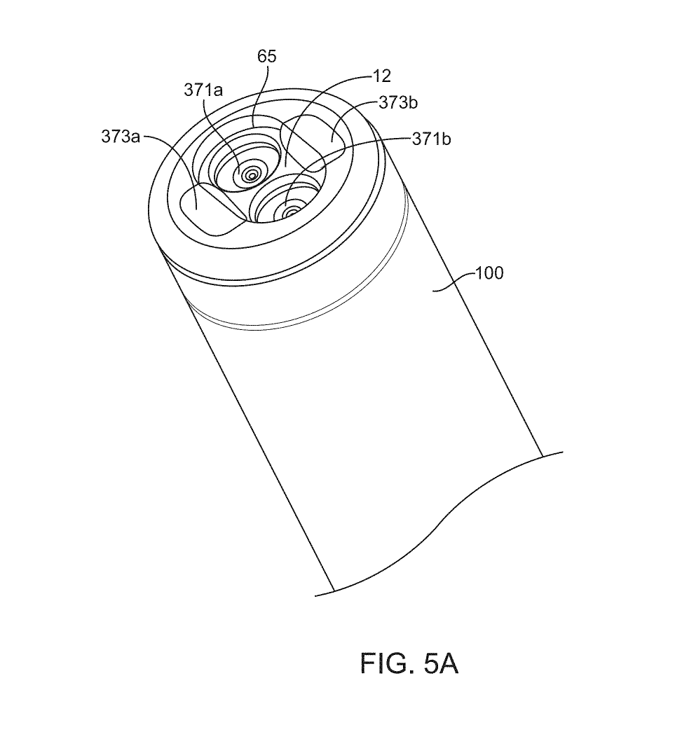

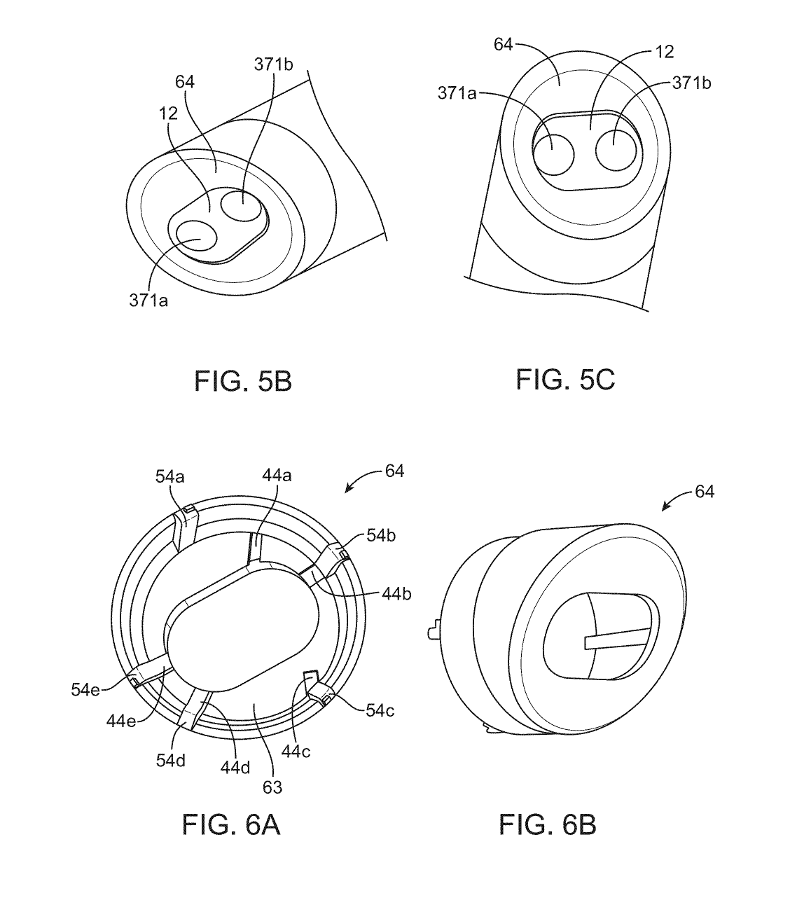

Referring to FIGS. 1 and 2, a laparoscopic sheath 100 (for use with an exemplary laparoscope 12) can include an elongate member 121 configured to ride over a shaft 227 of a laparoscope 12, a proximal end 101 configured to provide stability and registration of the sheath 100 on the laparoscope 12, and a plurality of channels 34a-e (see FIGS. 4 and 5) extending along the length of the shaft 121 of the sheath 100. Referring to FIGS. 5A-5B, in some embodiments, the sheath 100 can be configured to be placed over a binocular laparoscope having two lenses 371a,b and two light elements 373a,b. Moreover, in some embodiments, the sheath 100 can be configured to be used in conjunction with a robotic laparoscope.

### Characteristics of the Sheath and Sheath System

The laparoscopic sheath 100 can be designed to provide air flow and/or a surfactant through the channels 34a-e to clear the laparoscopic lens, as described further herein. Exemplary air flow systems are described in the '8014 application and the '7084 application, incorporated by reference above.

FIG. 1 shows an exemplary view optimizing assembly 1000 incorporating the laparoscopic sheath 100. As will be described in greater detail below, the view optimizing assembly 1000 facilitates intro-operative defogging, surgical debris deflection, and cleaning of a laparoscope lens during minimally invasive surgery while also maintaining visualizing of the surgical site. In some embodiments, the sheath 100 can be a single-use disposable laparoscopic accessory for use with the assembly 1000.

The view optimizing assembly 1000 can include the sheath 100 mounted over a laparoscope 12. The end of the sheath 100 can be sized and configured to match the size and configuration of the corresponding laparoscope 12, which can include either a blunt tip or an angled tip. The assembly 1000 further includes a tubing set 162 to connect the sheath 100 to an air supply 15, such as a carbon dioxide insufflator, and to a dispenser 72 of flushing fluid 74. A manifold 109 on the proximal end of the sheath 100 can connect the sheath 100 to the tubing set 162 through a quick exchange coupling 22. A bulb 174 can be connected to the tubing set 162 to provide a gas burst as necessary (e.g., to clear flushing fluid 74 off of the lens).

An exemplary connection between a manifold 109 and tubing set 162 is shown in FIG. 2. In some embodiments, such as embodiments where the sheath 100 is to be used with a robotic system, the tubing set 162 can have an angled connector 174 for gas/fluid inlets 111, 112. The angled connector 174 can extend, for example, at a 30-120 degrees angle from the axis of the inlets 111, 112, such as at a 90 degree angle. The angled connector 174 can be made, for example, of a stiff or hard material, such as a hard plastic. Such an angled connector 174 advantageously prevents the tubing 162 itself from kinking if pressed up against surgical tools or the patient.

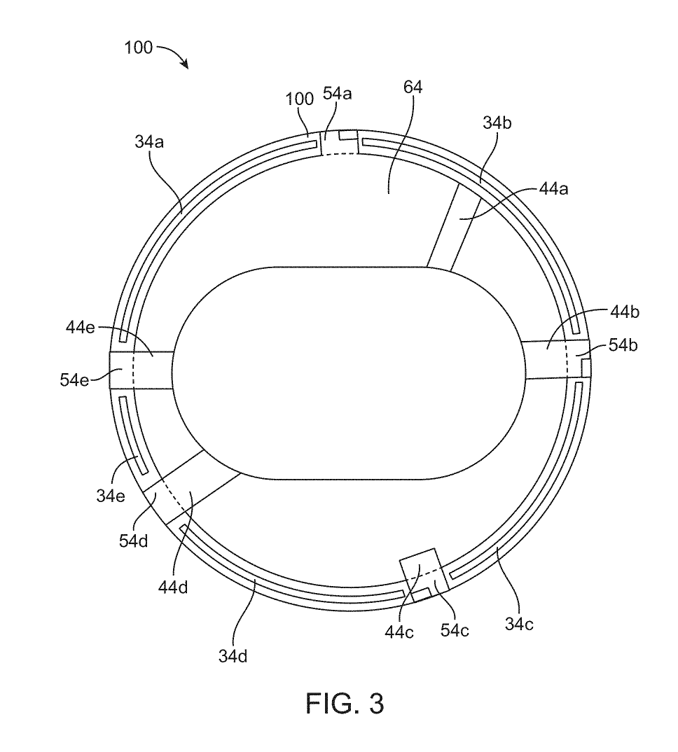

Referring to FIG. 3, the lumens 34a-e of the sheath 100 can extend the length of the shaft between the inner and outer walls of the shaft. In one example, there are five lumens 34a-e. The lumens 34a-e can be configured to deliver fluid and/or gas from the air and fluid supplies to the distal end of the sheath and ultimately over the end of the laparoscope. Further, each lumen 34a-e can extend a circumferential length that is optimized for the desired flow characteristics. Referring to FIG. 4, in one embodiment, for example where sheath 100 has an outer diameter of 0.417″ and an inner diameter of 0.335″, lumens 34a and 34b can each can extend a circumferential length of 0.258″, lumens 34c and 34d can extend a circumferential length of 0.225″, and lumen 34e can extend a circumferential length of 0.103″. The radial length of each lumen 34a-e can be 0.021″. In some embodiments, the smallest channel 34e can be configured to deliver fluid over the lens (e.g., for cleaning) while the larger channels are configured to deliver air over the lens (e.g. for debris removal and defogging). The sheath 100 can advantageously fit over an 8.5 mm robotic scope and through a 10 mm trocar.

Referring back to FIG. 3, the sheath 100 can include a deflector assembly 64 on the distal end thereof. The deflector assembly 64 can include including dividers 44a-e extending along an inner surface of the outer wall. The dividers 44a-e can extend substantially contiguous with a separation between the lumens 34a-a within the sheath 100. Further, the deflector assembly 64 can include stand-offs 44a-e on an inner face 64 of a distal face of the deflector assembly 64. The deflector assembly 64 can be configured to divert the flow of gas or fluid from the lumens 34a-e and over the lens.

Referring to FIGS. 5A-5C, in some embodiments, the sheath 100 can be configured to be placed over a binocular laparoscope 12 having two lenses 371a,b and two light elements 373a,b. The sheath 100 can include a deflector assembly 64 covering both light elements 373a,b while leaving the lenses 371a,b exposed. Thus, for example, an opening 65 in the distal face of the deflector assembly 64 can be oblong or oval in shape to accommodate the side-by-side lenses 371a,b. Moreover, an inner surface 63 of the deflector assembly 64 can be polished so as to allow the light to shine therethrough. Further, the deflector assembly 64 can project a predetermined distance beyond the distal end of the sheath 100, and thus also a predetermined distance beyond the lenses 371a,b of the laparoscope 12.

Referring to FIGS. 6A-6B, the stand-offs 44a-e on the inner-surface of the deflector 64 can serve to create a gap between the end of the scope and the distal face of the deflector 64. By doing so, an air flow path is created over the lens with an optimized velocity. In some embodiments, for example, the stand-offs 44a-e move the lens off of the inner surface 63 of the deflector 64 by 0.004″ to 0.008″, such as approximately 0.006″. Referring to FIGS. 5A and 6A, the stand-offs 44a-e are configured so as to avoid being positioned against the light elements 373a,b. Thus, while stand-offs 44a, 44b, 44d, and 44e extend all the way from the inner wall of the sheath 100 to the opening 65 in the deflector assembly 64, stand-off 44c can extend only part way from the inner wall of the sheath to the opening 65 to ensure that the stand-off 44c does not touch a light element 373. Likewise, while stand-offs 44b-e can extend from dividers 54b-e, stand-off 44a can be offset from divider 54a to ensure that stand-off 44a does not touch a light element 373a.

Referring again to FIG. 6A, the dividers 54a-e can be configured so as to be axially aligned with the separations between lumens 34a-e. The dividers 54a-e can also extend radially inwards so as to center the laparoscope when placed therein. Further, when a laparoscope is placed therein, the seal created between the laparoscope and the dividers 54a-e can continue the air flow pattern from the lumens 34a-e and distally down the deflector assembly 64.

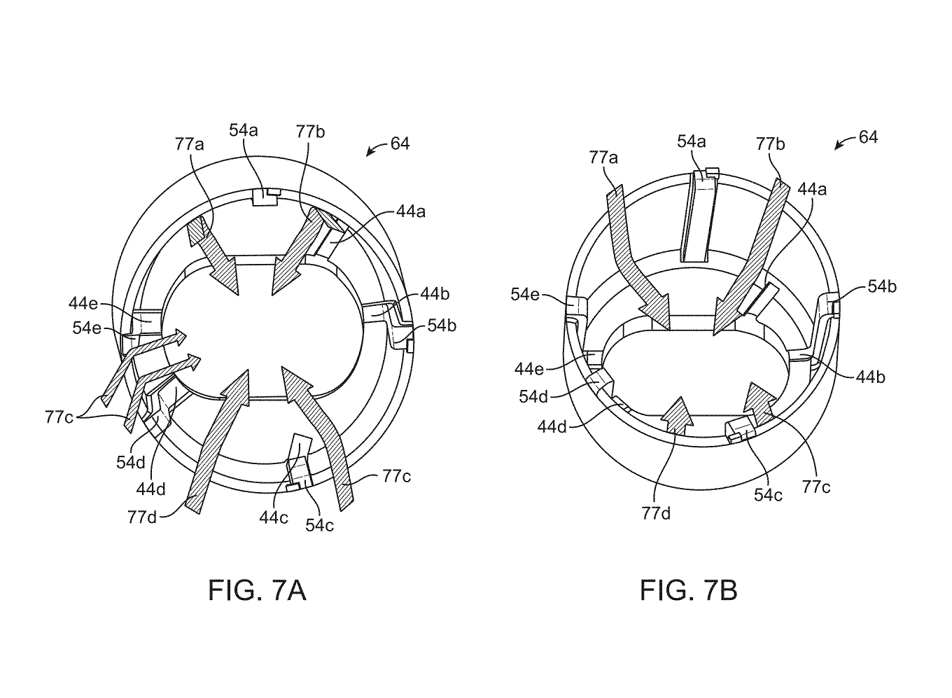

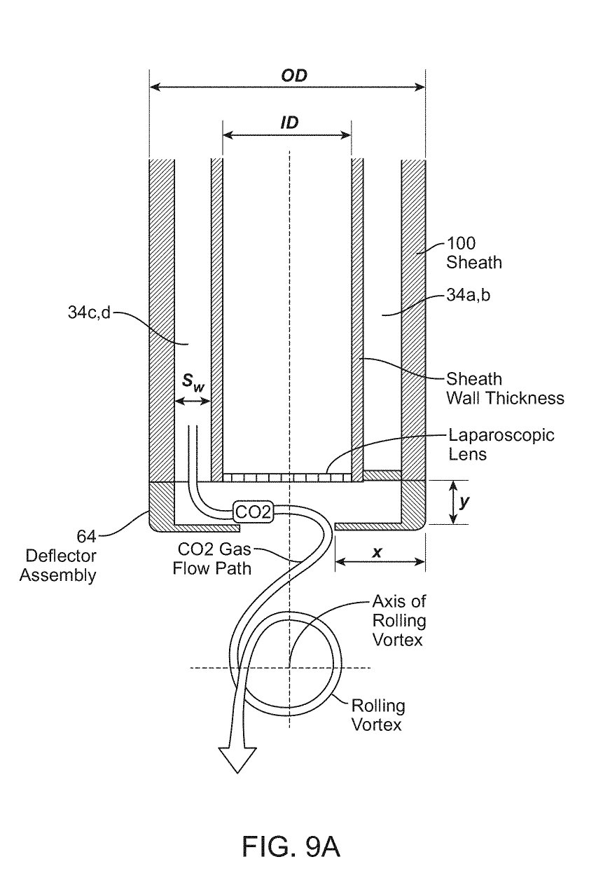

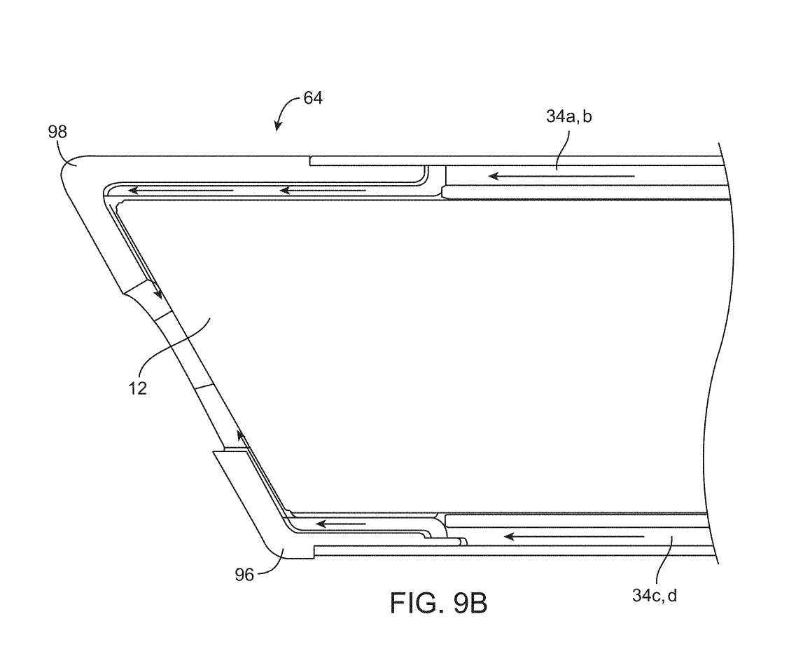

Referring to FIGS. 7A-7B, the combination between the dividers 54a-e and the stand-offs 44a-e are configured to create isolated air flow from each lumen. That is, the deflector assembly 64 can be attached to the sheath 100 such that the air or fluid flowing down the lumens 34a-e exits the lumens 34a-e and travels in a path between the dividers 54a-e/stand-offs 44a-e and the laparo scope itself (see also FIG. 9B). Air flowing out of lumens 34c and 34d (indicated by arrows 77c,d) can merge together once past the short stand-off 44c. Likewise, air flowing out of lumens 34a,b (indicated by arrows 77c,d) can merge together once past the divider 54a,b. However, fluid flow from channel 34e (indicated by arrows 77e) cannot mix with air flows 77a,b or 77c,d until it exits the deflector assembly 64 due to the dividers 54d-e and stand-offs 44d-e. Likewise, divider 54b and stand-off 44b separate air flows 77a,b from air flows 77c,d. As described further below, air flows 77c,d can have a higher velocity than air flows 77a,b to provide the desired lens cleaning characteristics.

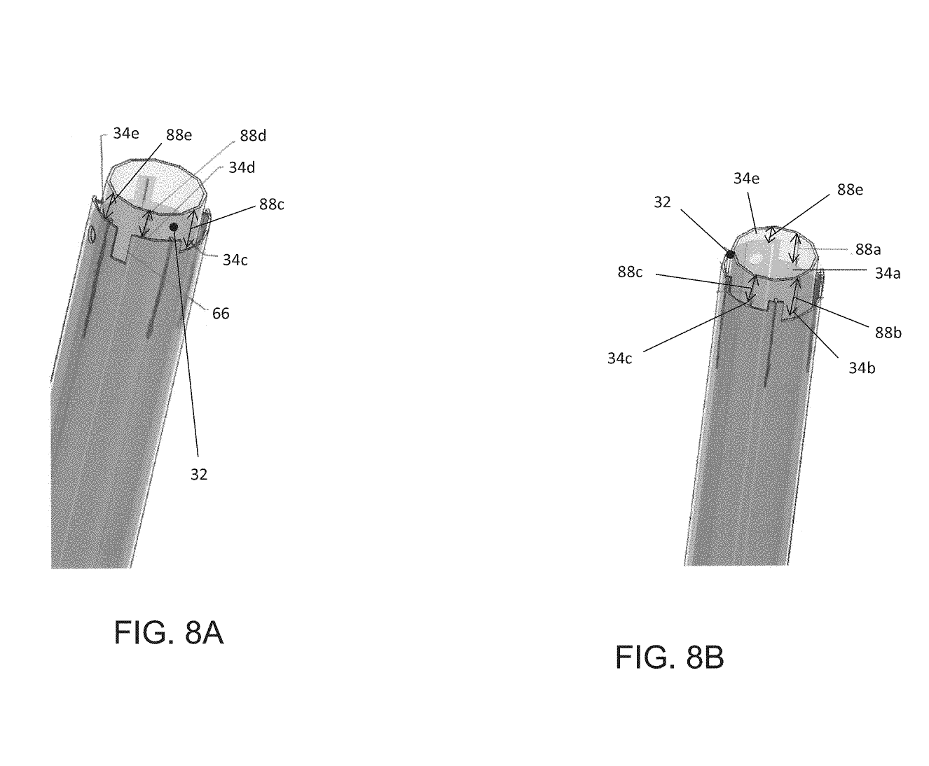

Referring to FIGS. 8A-8C, in some embodiments, the outer wall of the proximal end of the sheath 100, which is sized and configured to fit into the manifold 109 and registered using a keyway 66, can be reduced or trimmed to form cut-outs 88a-d for each gas-conveying lumen 34a-d. The cut-outs 88a-d provide space for the gas from inlet 32 to travel around the proximal tip of the sheath (e.g., in a plenum) and thereby down each of the lumens 34a-d. Cut-out 88a can have a length of 0.190″, cut-out 88b can have a length of 0.190″, cut-out 88c can have a length of 0.140″, and cut-out 88d can have a length of 0.090″. The positioning of the lumens 34a-e, in addition to the shape and size of the cut-outs 88a-d, can be configured and optimized so as to control the speed of air flow through the lumens 34a-d. Thus, adjacent lumens 34c and 34d can be configured to have higher gas flow than lumens 34a and 34b. This is because lumens 34c,d are closer to the gas inlet 32 than lumens 34a,b. Thus, lumen 34c (which is closest to the inlet 32) can have the highest flow, lumen 34d can have the second highest flow, and lumens 34a,b can have the lowest flow. Further, the proximal end of the fluid lumen 34e can be plugged so as to avoid mixing of the fluid from the channel with the air plenum.

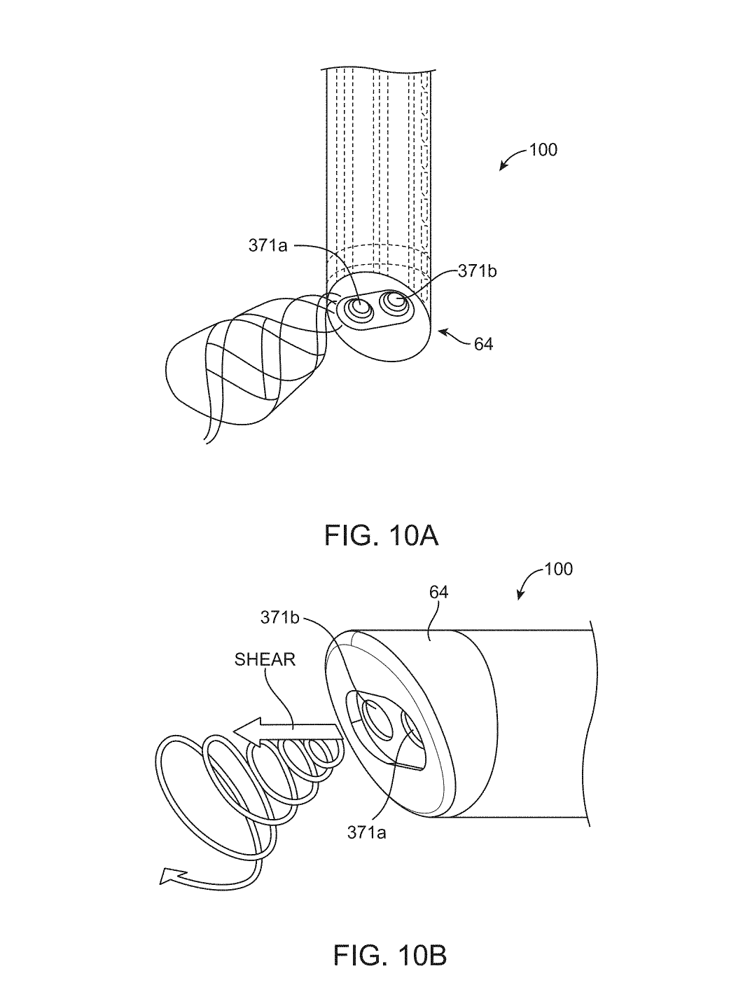

Referring to FIGS. 9A-9B, the difference in velocities of gas flowing through the lumens 34c-d can form a rolling vortex as it exits the opening 65 of the deflector assembly 64. That is, as shown in FIGS. 9A and 9B, the deflector assembly 64 overhangs the laparoscopic lens (such as binocular lens) by a prescribed transverse distance, defining a deflection width X, sufficient to change the direction of gas flowing axially through lumens 34a,b of the sheath 100 (i.e., along the axis of the laparoscope shaft) into a non-axially, transverse path across the laparoscopic lens. The distance of the deflection width X does not extend to the point that is obstructs the field of the view of the laparoscopic lens. As also shown in FIGS. 9A and 9B, the deflector assembly 64 projects axially beyond the distal terminus of the sheath 14 by a prescribed axial distance, defining an air channel distance Y, sufficient to maintain the gas flowing along the path bounded by the deflection width X at a distance sufficiently close (proximal) to the laparoscopic lens. Further, as shown in FIG. 9B, if a deflector assembly 64 is designed for an angled tip, it can be positioned such that the high flow lumens 34c,d are positioned at the heal 96 while the low flow lumens 34c,d are positioned at the nose 98. In this way, the vortex stems from the heal 96 (see also FIG. 10A).

Together, the deflection width X and the channel distance Y define the pneumatic characteristics of the deflection assembly 64. At the desired minimum flow rate (e.g., greater than 0.1 L/min or approximately 1 L/min for defogging and 2-3 L/min for deflection), the pneumatic characteristics create a flow path that diverts gas from the lumens 34c,d at the desired flow velocity across the laparoscopic lens toward the facing side of the deflection assembly 64 (see FIGS. 7A and 7B). A rolling vortex can be created that extends across and beyond the laparoscopic lens. The rolling vortex creates an air curtain across the lens sufficient to defog the lens.

Referring to FIGS. 10A-10B, a rolling vortex is formed that creates a “vortex shearing” effect across and beyond the laparoscopic lens, disrupting the ambient atmosphere at or near the tip of the sheath 100 to create a clear zone that extends across and beyond (by approximately 0.25 inch or more) the plane of the lens. The clear zone created by the vortex shearing effect prevents fogging, as well as deflects smoke and surgical debris away from the viewing field of the laparoscopic lens during surgery. The rolling vortex is positioned beyond the lens with a sufficient air velocity to attract the particles away from the lens and thereby avoid entrainment or deposition of particles on the lens. To avoid entrainment, it is desirable that the vortex attracts particles away from the lens in a direction (when looking down the scope in a distal direction) toward a 3 O'clock (090) or Right Side or in a direction (also when looking down the scope in a distal direction) toward a 9 O'clock (270) or Left Side. The rolling vortex can spiral about an axis that is approximately transverse the axis of the sheath (FIGS. 10A-10B show at a slightly lower angle for clarity). Gas eventually exits the rolling vortex in a flow path that extends generally parallel to the axis of the sheath 100, carrying the particles with it. When viewed through a camera screen, the vortex can form in the lower left hand corner opposite the fluid lumen 34e.

Referring to FIG. 10B, the vortex can entrain smoke and debris that is directed to the lens as well as any smoke that is circulating in the abdominal cavity and passes in front of the tip/lenses 371a,b. The shearing stream can push debris and smoke entrained in the vortex away from the tip and lenses 371a,b. Further, the vortex is designed to direct gas upward across the lenses 371a,b.

Referring back to FIGS. 9A and 9B, while lumens 34c and 34d primarily form the vortex, lumens 34a and 34b can help dry the lens when cleaning solution is applied through lumen. That is, as mentioned above, the tubing set 162 can be connected to a source 72 of sterile liquid 74, such as saline, sterile water, or dioctyl sulfosuccinate salt, such as dioctyl sodium sulfosuccinate (DSS) (also referred to as docusate sodium), docusate calcium, or docusate potassium. When the quick exchange coupler 22 is connected, operation of the syringe 74 directs bursts of the sterile liquid through the lumen 34e in the sheath 100 to the deflector assembly 64 at the distal end of the sheath 100. In this arrangement, the deflector assembly 64 is also sized and configured to direct the burst of sterile liquid in a desired path across the laparoscopic lens. The bursts of sterile liquid serve to flush debris off the end of the lens that may eventually accumulate, thereby cleaning the lens. Thereafter, bursts of air supplied by the lumens 34a-e to the deflector assembly 64 serve to clear residual fluid droplets off the lens to maintain an acceptable view.

In some embodiments, the central lumen of the sheath through which the laparoscope extends also allows gas to flow therethrough. However, the central lumen can be sealed proximally and flow limited so that most of the gas is delivered by the lumens 34a-e.

The sheath 100 described herein provides several advantages when used to defog and clean a laparoscopic lens. The sheath includes two adjacent lumens with high flow rates that form a vortex for defogging and debris removal at the end of the laparoscope tip. The high gas flow is directed upwards across the tip of the laparoscope. All of the lumens work together to effectively dry cleaning fluid placed on the lens. The tip opening is close to the viewing window, but does not create glare or reflection. Further, all of the gas flow provided from the manifold is deflected into a lumen or down the center of the sheath, thereby providing precise control of the flow. Fluid flow is isolated from gas flow, thereby preventing bubbling or mixing of the fluid and gas. Light reflection and glare is minimized by optimizing the position of the opening in the viewing window of the lens, the edges of the opening, and the polish level of the tip. Further, the opening size and shape of the deflector are designed to be as close as possible to the viewing angle of the scope. The distal end of the sheath are configured such that any ledge portions at the distal end overlap light coming out of the laparoscope but not the optics. By providing optimized and precise gas and fluid flow, the vortex and shear effect are able to keep the fog and debris away from the lens. Without such control, smoke and debris can circulate in front of the lens, looking like a cloud, and/or debris can reach the lens either directly or through entrainment of the chaotic gas flow.

It is to be understood that the sheath 100 can be modified so as to work with a variety of different imaging systems.

### Registration and Alignment

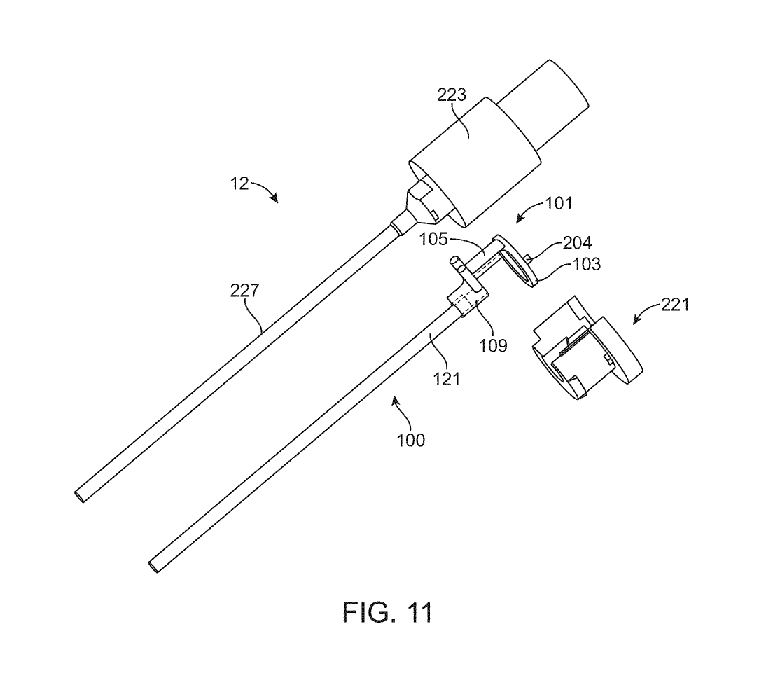

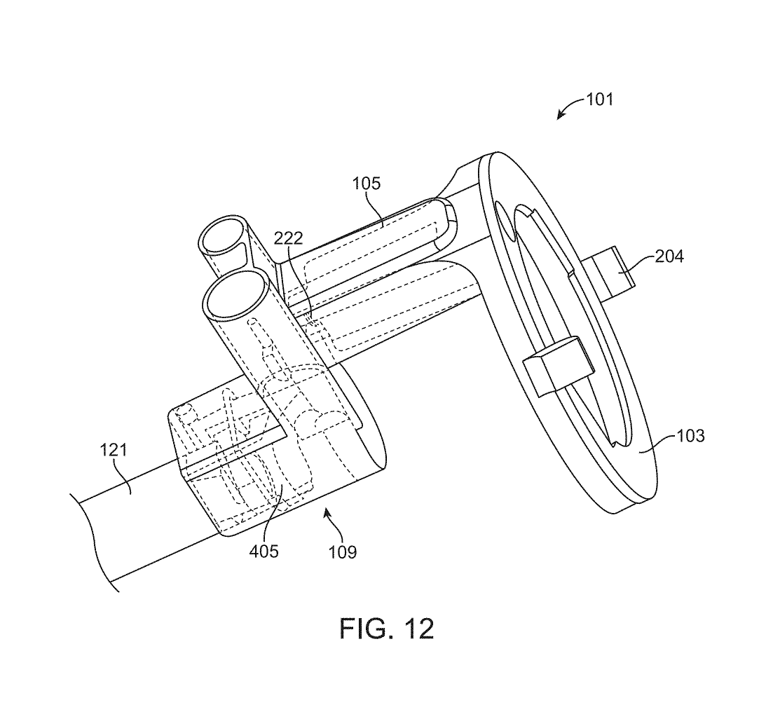

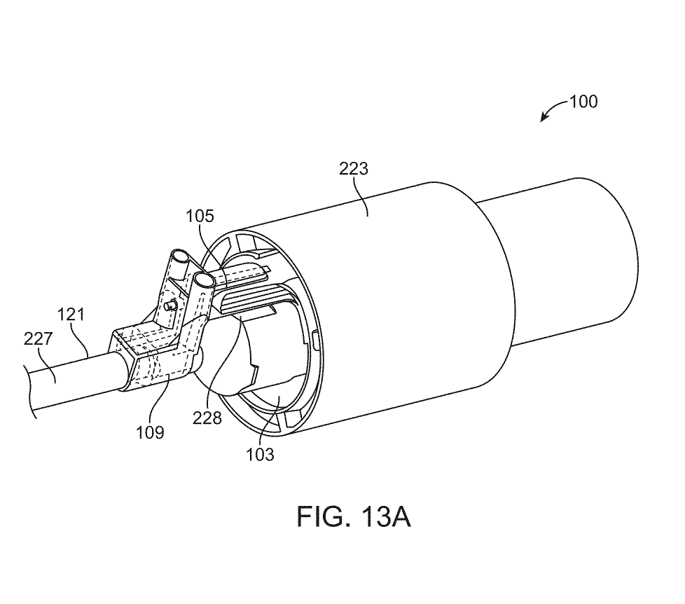

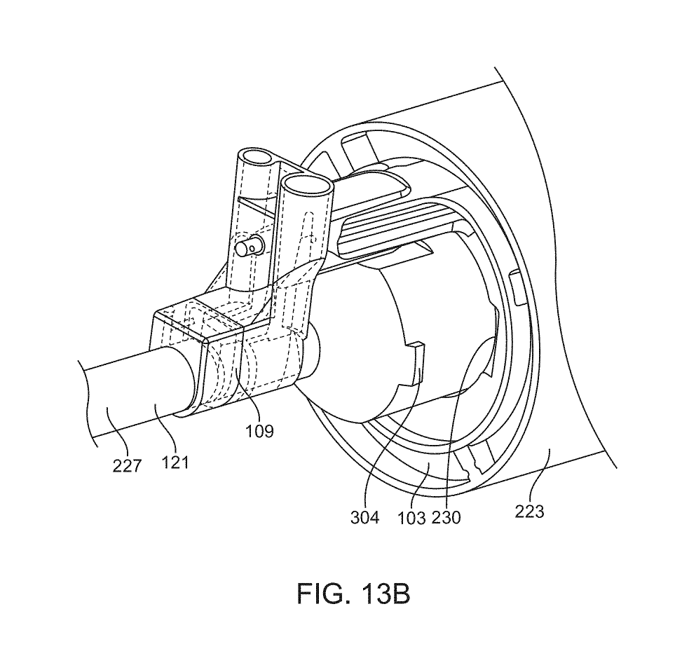

Referring to FIGS. 11-13B, the sheath 100 can include a registration and alignment feature configured to maintain the relative positioning of the sheath 100 and the scope 12. As shown best in FIG. 12, the proximal end 101 of the sheath 100 can include a manifold 109, a resilient member 105, and a connector piece 103. The connector piece 103 can be configured to mate between two features of the laparoscope and/or camera system and/or other auxiliary equipment used in the medical procedure. For example, connector piece 103 can be a flat annular ring or partial ring configured to snap in place between two features. Shapes other than circular may be used in the connector piece 103 and rings of even smaller dimensions may be utilized. As shown in FIGS. 13A-13B, the sheath 100 can be placed over a laparoscope such that the elongate member 121 fits over the shaft 227 of the laparoscope while the proximal side of the connector piece 103 sits flush against the distal surface of the proximal portion 223 of the laparoscope (e.g., within a milled track).

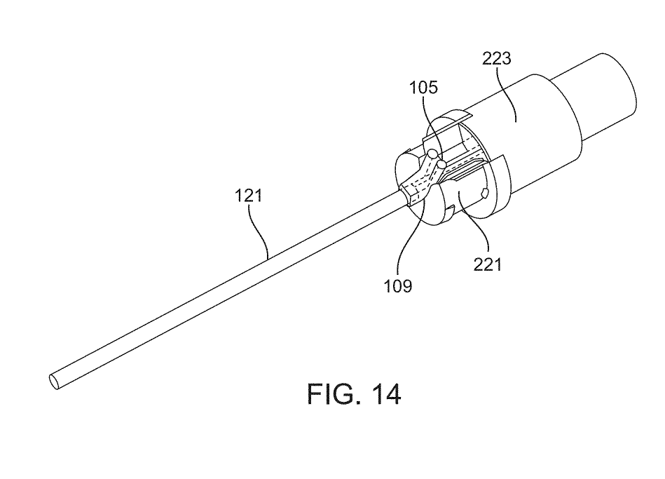

As shown in FIG. 14, a mating portion of the laparoscope or camera, such as a camera mount 221, can then be placed on the distal side of the connector piece 103 to lock the connector piece 103 in place. The camera mount 221 can include a similar milled track within which the distal side of the connector piece 103 can sit. The connector piece 103 can thus be sandwiched between the proximal portion of the laparoscope 223 and the camera mount 221. In some embodiments, if there is no milled track in the laparoscope and/or the camera mount 221, an adaptor can be used to provide the necessary attachment between the laparoscope and the camera mount 221 while still allowing for the placement of the connector piece 103 of the sheath therebetween.

The connector piece 103 can further include keying features, such as cut-outs or notches, to help it slide over and/or mate with portions of the laparoscope. For example, the inner perimeter of the connector 103 can include a flattened portion that slides over a flattened portion 228 (see FIG. 13A) of the proximal portion of the laparoscope 223 to provide for initial alignment of the scope and the sheath. Likewise, the inner perimeter of the connector piece 103 can include cut-outs 230 (see FIG. 13B) to match extensions 304 (see FIG. 13B) on the proximal portion 223 of the laparo scope.

The connector piece 103 can help hold the sheath 100 in place axially (i.e., such that as the laparoscope is moved proximally and distally, the sheath 100 simultaneously moves proximally and distally). Moreover, in some embodiments, the connector piece 103 can help prevent rotation of the sheath 100 relative to the laparoscope either through a tight friction fit or through keying features thereon.

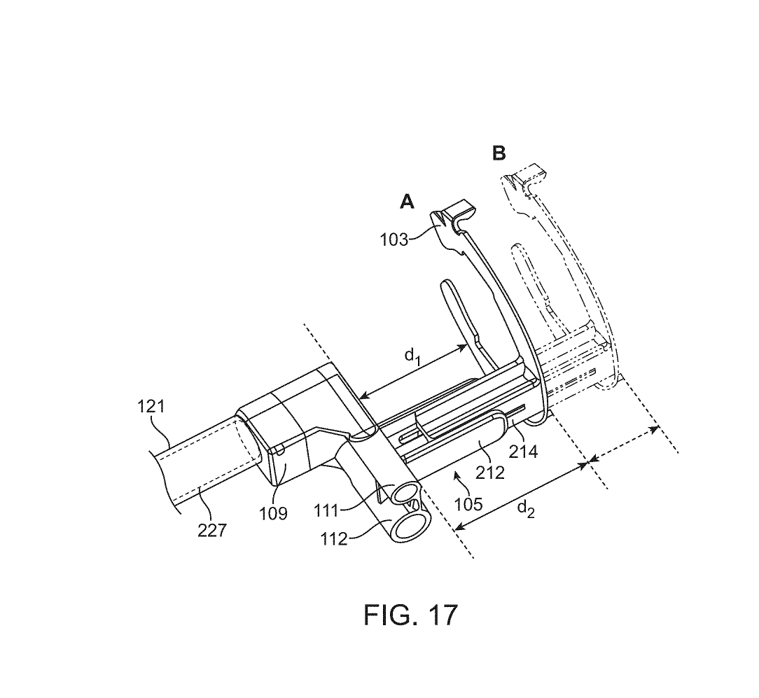

The resilient member 105 of the sheath 100 can be between the connector piece 103 and the elongate body 121 and can provide registration of the sheath 100 relative to the laparoscope. The resilient member 105 can be, for example, a spring, a pneumatic actuator, or strip of elastic material, such as rubber. FIG. 17 is a perspective view of an embodiment of a sheath 100 having a resilient member 105 connected to a manifold 109. The manifold 109 has a pair of inlets 111 and 112, is connected to the sheath with a central opening to receive the scope as described in the '7084 application and the '8014 application. At the point of scope insertion into a sheath, proximal portions of the sheath and scope may form a friction fit. However, other misalignments may exist or, in use, the scope or sheath may move out of registration. Advantageously, a biasing force may be produced by displacement of a resilient member 105. The biasing force may then be used to maintain and ensure registration of the scope tip-sheath tip in use as described further below.

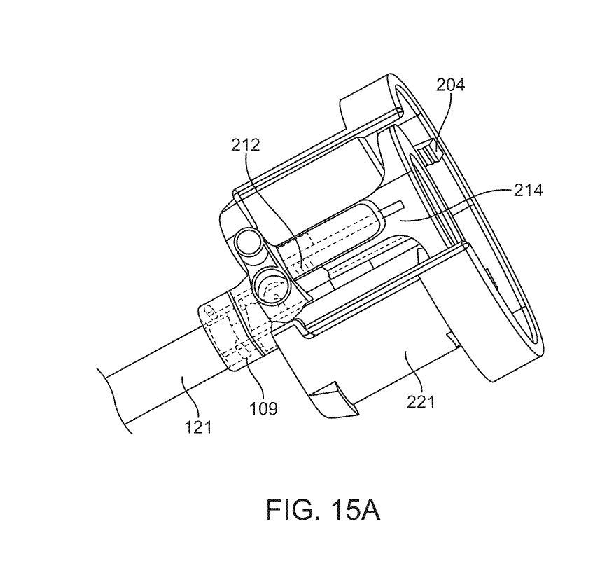

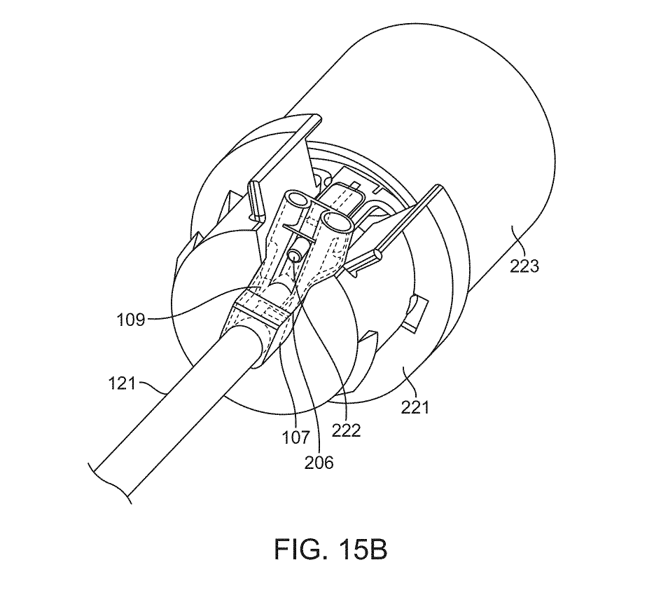

The resilient member 105 illustrated in the embodiment of FIG. 17 includes a base 212 and a sliding mount 214 (see also FIG. 5A). The proximal end of the sliding mount 214 is attached to the connector piece 103 described above. The sliding mount 214 movement places the connector piece 103 into different positions (positions d1 and d2 are shown). Further, a spring 222 (see FIGS. 12, 15B) can extend along part of all of the length of the resilient member 105 to provide the necessary resilient force.

The resilient member 105 can take on a number of different embodiments and can include, for example, a spring, a rubber band, a hydraulic or pneumatic piston or other suitable element to modulate the displacement and position of the sliding mount/connector piece 103 to the manifold 109. As a result, the resilient member 105 can be adapted and configured to provide a controllable or desired amount of bias. As described elsewhere herein, when the connector piece 103 is secured with a scope in the sheath, displacement of the resilient member 105 is translated into a force to pull the sheath 100 proximally and into registration with the scope (i.e., tip of sheath and tip of scope are in a desired alignment and position). The same force can also be used to overcome any force that the laparoscope or laparoscopic seal might place on the sheath 100 during use. For example, the resilient member 105 can be configured to provide 8-12 lb of resilient force, such as 10 lb of force. This force can advantageously be enough to maintain the position of the distal end of the scope relative to the distal end of the sheath for robotic laparoscopy applications. For manual laparoscopy applications, the resilient member can be configured to have a lower spring force, such as 1-5 lb, e.g. approximately 2 lb.

In one exemplary embodiment, a scope or surgical instrument is inserted into the sheath lumen. As the scope or instrument is advanced into position, a proximal portion of the scope or instrument displaces the connector piece 103 (i.e., from d1 to d2) and as a result, a biasing force produced by displacement of the resilient member. The biasing force then acts to ensure registration of the scope tip-sheath tip in use. In another exemplary embodiment, a scope or surgical instrument is inserted into the sheath lumen. As the scope or instrument is advanced into position, a proximal portion of the scope or instrument either does not contact the connector piece 103 or only insubstantially displaces the connector piece 103 to generate a desired or adequate biasing force. In this circumstance or in other embodiments, a coupling or mating device may be suitably employed to displace the connector piece 103 or engage the resilient member to provide an appropriate biasing force. Still further, a coupling may include one or more features, for example, to facilitate mating with one or more surgical mounts or guides. Exemplary surgical mounts or guides include height adjustable mounts, articulating mounts, motor driven mounts or surgical robotic actuators.

In still another embodiment, the dimensions and operation of the base, sliding mount, resilient member and connector piece 103 are selected for an appropriate size and fit onto or between two existing components in a surgical system using the sheath. For example, the above described components may be adapted and configured so that the connector piece 103 is positioned between a portion of a scope 223 and a portion of a camera module, such as a camera mount 221. In still another embodiment, the above described components are adapted and configured for placement between two components that snap together or screw together in which case the connector piece 103, or other components are modified so as engage with the components being joined without interfering with the particular joining operation or function. For example, the connector piece 103 may be dimensioned to act as a washer that fits between the two joining components.

Thus, during placement and locking of the sheath 100 over the laparoscope, the resilient member 105 can ensure automatic registration and placement/locking of the distal end of the laparoscope with the distal end of the sheath 100. As the sheath 100 is pushed into place over the laparoscope, the resilient member 105 will stretch or expand the appropriate amount to allow for an exact fit of the connector piece 103 relative to the laparoscope, thereby ensuring a tight axial fit of the scope relative to the sheath.

The sheath 100 can further include a separate keying mechanism 107 to help prevent rotation of the sheath 100 relative to the laparoscope. For example, the keying mechanism 107 can be beveled or grooved. In embodiments where the sheath 100 is used to provide air flow and/or a surfactant therethrough, the keying mechanism 107 can be part of a manifold 109 (see FIGS. 15A-15B). A gas and fluid inlet manifold is illustrated and described in the '7084 application and can modified as described herein to be used as manifold 109. In some embodiments, one or more features are added to or formed in a portion of the manifold 109 to assist in providing one or more of registration and stability between a sheath and a surgical scope. In one aspect, one or more sides of the manifold is beveled or has facets to cooperatively mate with one or more corresponding facets or features in another component in the surgical field or system in use.

Referring to FIGS. 15A-15B, the keying mechanism 107 can be formed on the opposite side of the manifold 109 as the inlets 111, 112. The keying mechanism 107 can be a beveled portion of the manifold 109 that is configured to precisely mate with a corresponding slot 206 on the camera mount 221. The slot 206 on the camera mount can be an asymmetric cut-out through an otherwise primarily circular camera mount 221. The slot 206 can, for example, be further used in a robotic system by allowing for the removal of the elongate surgical scope during use without having to pull the scope axially through the mount. For example, referring to FIG. 8, the slot 206 can advantageously allow the shaft 227 to be pulled laterally from the camera mount 221 (i.e., in a direction to uncouple the manifold from the camera mount) without requiring the entire proximal portion 223 and elongate member 227 to be pulled proximally (axially) through the camera mount 221.

The keying mechanism 107, when mated with corresponding features on the laparoscope or camera (such as the slot 206 on the camera mount 221), can advantageously prevent the laparoscopic sheath from rotating during use. In other embodiments, the keying mechanism 107 can engage a corresponding component via friction fit, a latch, a clip, a belt or other suitable restraint consistent with the other form factors described herein. Examples of other components include for example a portion of a surgical instrument, a portion of a laparoscope, a separate coupling attachment or a coupling attachment used in combination with a surgical stability system, articulating stability system, an electromechanical stability system or a robotic surgical actuator.

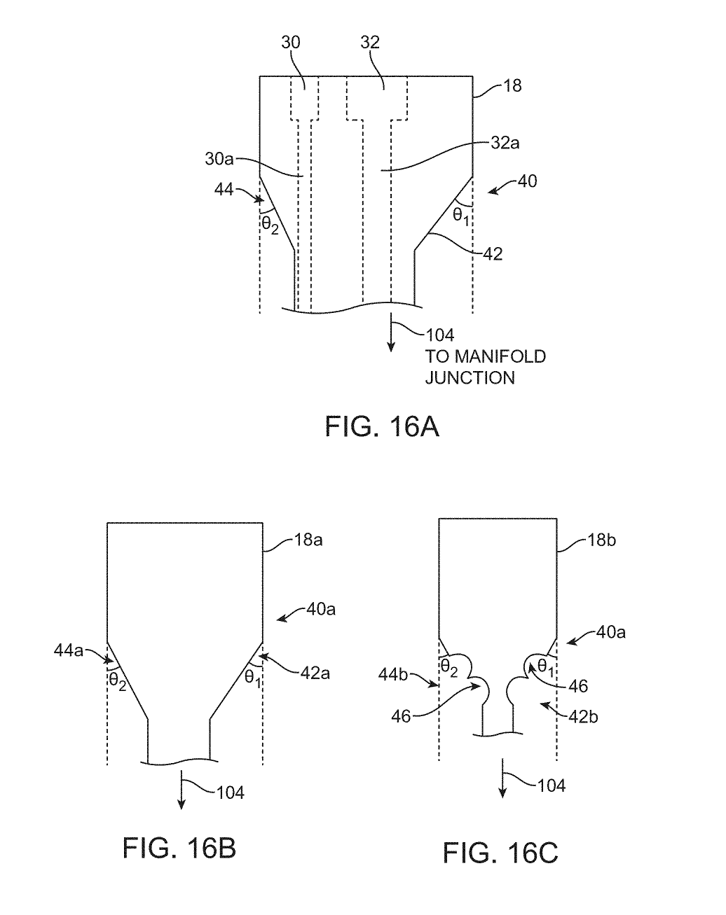

FIGS. 16A-16B illustrate section views of a manifold embodiments 18, 18a and 18b respectively that can be used as manifold 109 and modified to include one or more features to assist in providing one or more of registration and stability between a sheath and a surgical scope. Each manifold embodiment includes the inlets 30, 32 and channels 30a, 32a shown in phantom in FIG. 16A (but not shown in the views of FIGS. 16B and 16C). In general, there is a surface 42 adjacent the larger passage 32a and a surface 44 adjacent the smaller passage 30a. In the embodiment of FIG. 16A, the surface 42 is inclined from the side of the manifold at the angle θ1 and the surface 44 is inclined on another side at an angle θ2. In this illustrative embodiment, the θ1 is greater than θ2 and the length of surface 42 is longer than surface 44. In contrast to the different angles and lengths of FIG. 16A, FIG. 16B illustrates an embodiment where the angles θ1 and θ2 are equal as are the lengths of surfaces 42a and 44a. In contrast to the smooth surfaces 42, 44, 42a, 44a embodiments of FIGS. 16A and 16B, FIG. 16C illustrates surfaces 44b and 42b that include features 46 in the surface. While FIG. 16C illustrates features 46 that are semi-circular and of about the same size, spacing and orientation, other feature 46 embodiments are possible including different shapes (e.g., linear, zigzag, oblong or oval), size and number (fewer and larger or smaller and more) and orientations (different angular relationship of the feature 46 to the surface 42/44. While the various manifold embodiments 18, 18a and 18b have been simplified in FIGS. 16A-16C to provide details of the features described above, it is to be appreciated that these manifold embodiments are provided with the other appropriate functional aspects and engineering details as described in the '7084 application and the '8014 application.

In some embodiments, additional attachment features, such as tabs 204 (see FIG. 15A) can be used to further enhance the attachment between the connector piece 103 and the camera mount 221. The tabs 204 can prevent the sheath 100 from sliding longitudinally down the laparoscope, can reduce the force applied to the camera mount 221 to make it easier to remove the laparoscope and/or the sheath 100 from the camera mount 221, and can limit rotation of the sheath 100 when not inserted in the camera mount 221.

In some embodiments, the manifold 109 can further include a compression fitting 405 therein (see FIG. 12). The compression fitting 405 can be an annular ring around the inner lumen of the manifold at the connection between the manifold 109 and the shaft 227 of the laparoscope. Advantageously, the resilient member 105 can provide sufficient biasing force to ensure activation of the compression fitting 405. The compression fitting 405 can ensure that air does not escape back up through the shaft 111 during use of the sheath 100 and the laparoscope.

In use, as shown best in FIG. 14, the sheath 100 can slide over the elongate portion 227 of a laparoscope from the distal end to the proximal end. When the sheath 100 is placed completely over the laparoscope (i.e. such that the distal end of the laparoscope sits against the distal end of the sheath, e.g., against stand-offs on the inside of the distal end of the sheath), the elongate member 121 of the sheath 100 can be locked in placed relative to the elongate member 227 of the laparoscope, such as through a fiction fit with the manifold 109. In such a position, the connector piece 103 will sit close to or against the proximal portion 223 of the laparoscope. The camera mount 221 can then snap against the proximal portion 223 of the laparoscope, forcing the resilient member 105 into an extended position and catching the connector piece 103 therebetween. This connection of the connector piece 103, in conjunction with the keying mechanism 107, can provide stability and registration of the sheath relative to the laparoscope. By using the stability and registration features described herein, the need for bulky and cumbersome locking mechanisms, knobs, or alignment features can be avoided, thereby providing for a low-profile attachment mechanism.

Incorporation with Robotic System

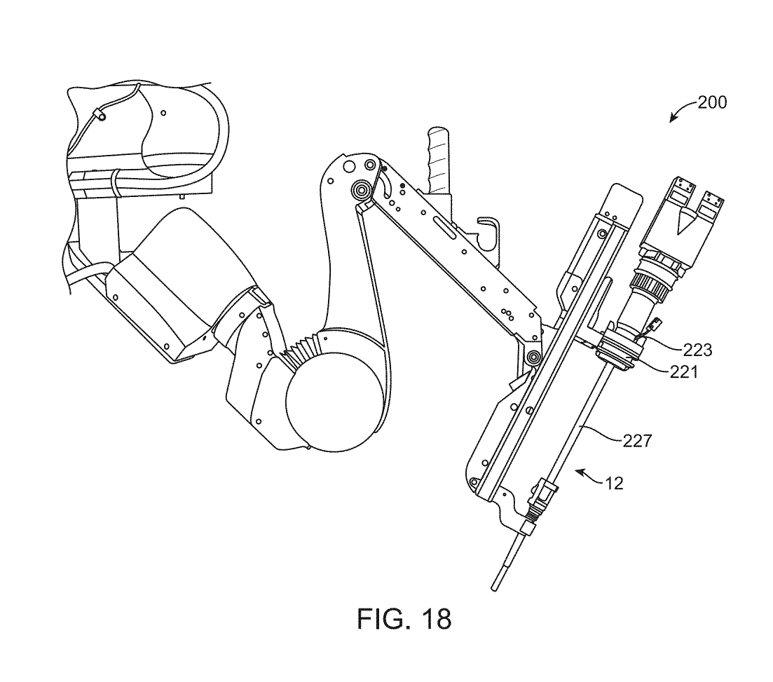

In some embodiments, the laparoscopic sheath described herein can be used with a laparoscope connected to a fixed stand, a single-joint system, or to an articulating robotic arm. An exemplary articulating robotic arm 200 with which the sheath 100 can be used is shown in FIG. 18. The connector piece 103 could be connected, for example, between the proximal portion of the laparoscope 223 and the camera mount 221 of the robotic arm shown in FIG. 8. The low-profile attachment mechanisms described herein can advantageously allow for use of the laparoscope with standard or prefabricated robotic systems, which often have limited room for additional features (such as registration or alignment knobs or forks).

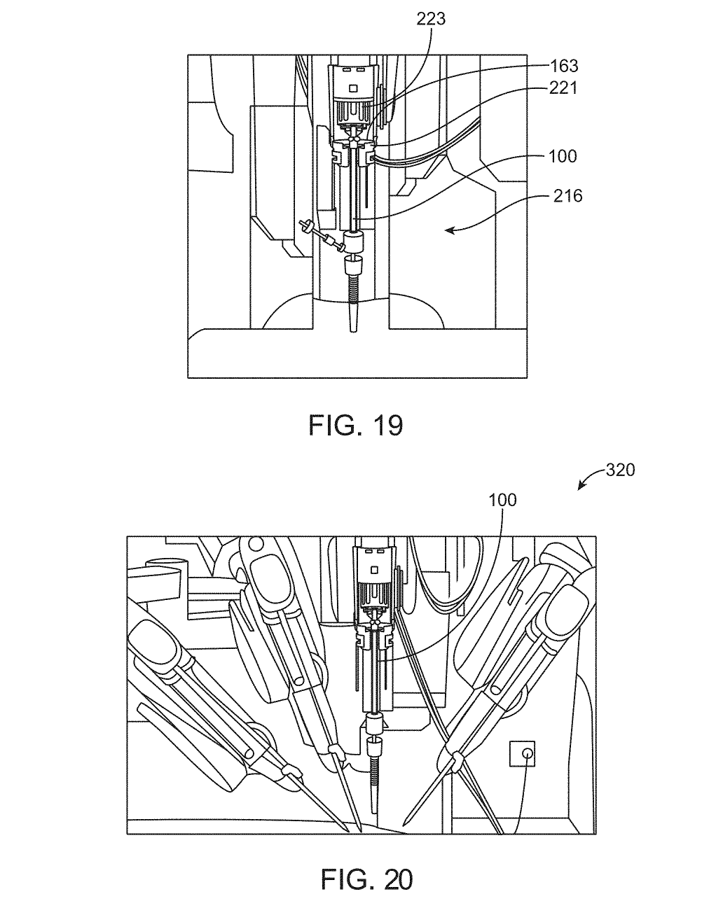

FIG. 19 shows the sheath attached to a laparoscope 12 of a robotic surgical system. The connector piece 103 extends between the proximal portion 223 of the laparoscope 223 and the camera mount 221. As shown in FIG. 20, the sheath 100 can be used on a single arm, e.g., the laparoscopic arm, of a robot 320 including multiple robotic arms.

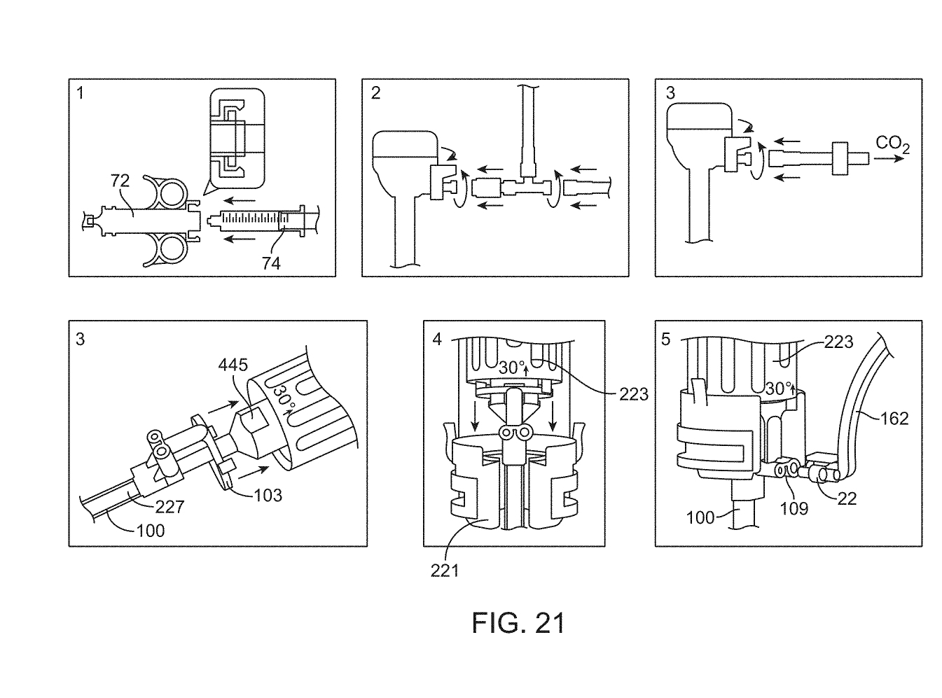

A schematic diagram of a method for connecting the sheath 100 to a laparoscope 12 of a robotic surgical system is shown in FIG. 21. At step 1, fluid 74 can be loaded into a syringe 72. At step 2, insufflation tubing can be connected to a trocar port. At step 3, a vent can be attached to the trocar port. As shown in Step 4, the sheath 100 can be slid in a proximal direction over the laparoscope shaft 227 until the connector piece 103 fits snugly over the proximal portion 223 of the laparoscope (as shown, the cut-out portion of the connector 103 can have a shape that mates over an extension 445 the proximal portion 223). A marker (such as the 30 degree marker shown in FIG. 21) can help indicate the correct orientation of the sheath relative to an angled tip. As shown in Step 5, the camera mount 221 can then be slid proximally over the sheath and then locked into place with the proximal portion 221 of the laparoscope 223, thereby catching the connector 103 therebetween. At step 6, the tubing set 16 can be attached to the manifold 109, which is exposed through the cut-out 229 in the camera mount 221.

### Control

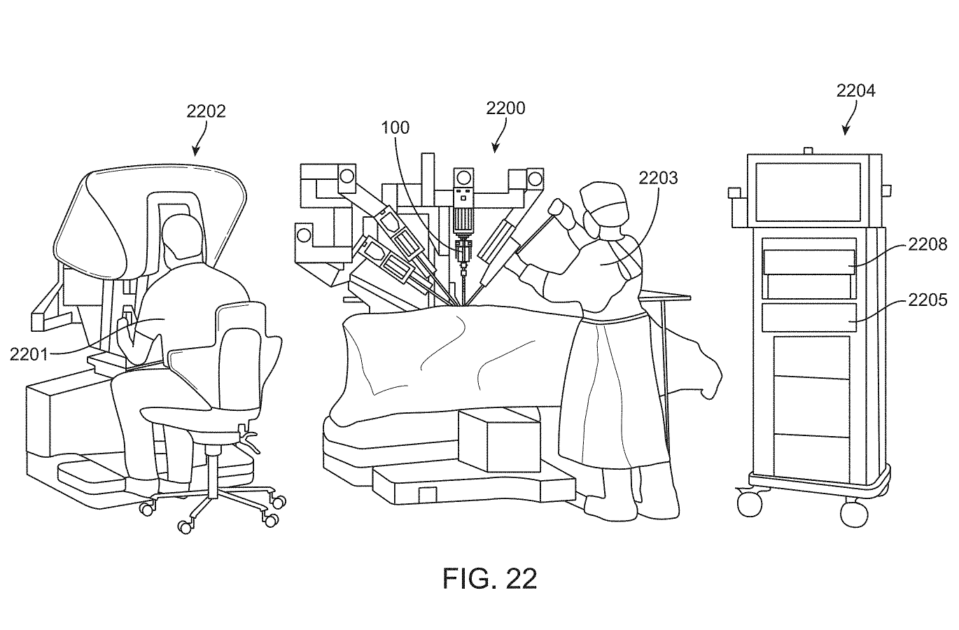

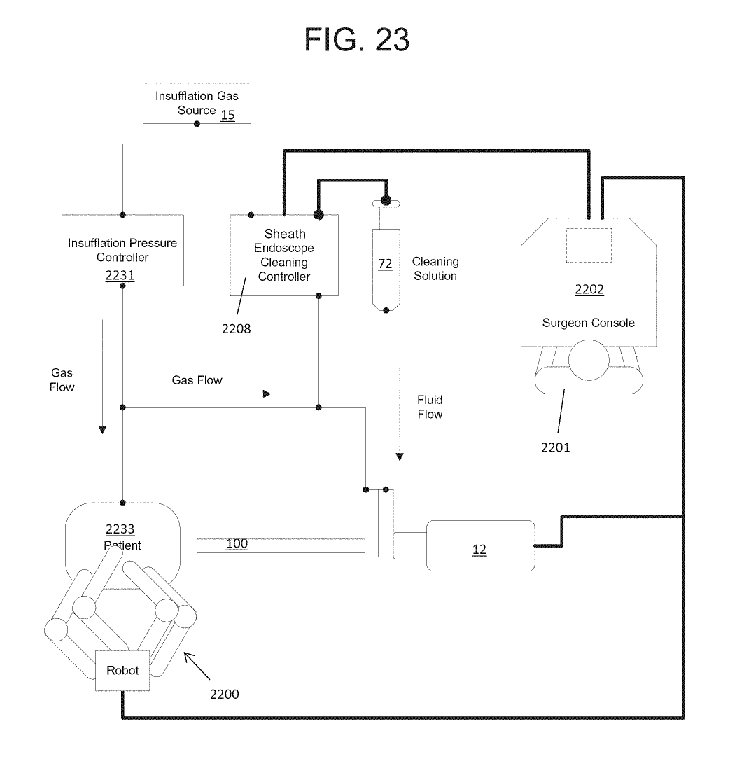

Referring to FIGS. 22 and 23, in some embodiments, control of the sheath 100 can be automated by the controllers used to otherwise control the robot. For example, as shown in FIG. 22, in one embodiment, there can be a console 2202 at which the surgeon 2201 sits to control the robot 2200 and a control tower 2204 configured to house the controller 2205 for the robot 2200. The controller 2208 for the sheath 100 can likewise be located in the tower 2202 and can be configured to activate the constant air supply, air burst, and fluid delivery. In some embodiments, the controller 2208 for the sheath can be easily insertable into or removable from the tower 2202. Further, the surgeon can activate the controller(s) 2205, 2008 with the console 2202.

To prepare the sheath 100 for use with the robot 2200, the medical provider 2203, such as a nurse, can load the dispensers of fluid onto the system, such as attach it to the tubing of the sheath assembly. The tubing can be primed, such as by activating a button on the controller 2204.

An exemplary system diagram is shown in FIG. 23. As shown in FIG. 23, the surgeon 2202 works at the console 2202 to send instructions to both the robot robotic controller 2205, the laparoscope 12, and the sheath controller 2208. The sheath controller 2204 controls the release of gas from the air source 15, such as an insufflator which can be regulated through an insufflation pressure controller 2231 and provided to the patient 2233 (for insufflation) and to the sheath 100 (for clearing of the laparoscope 12).

A detailed schematic of the controller 2204 is shown in FIG. 24. The controller 2208 can, for example, include a syringe tray holder 2404 configured to hold one or more syringe dispensers 72. A plunger drive bar 2404 can be coupled with the plungers of the syringe in order to dispense fluid to a fluid lumen 34e of the sheath 100 when activated by a servo motor 2406. A valve mechanism can ensure that fluid is not released from the lumen 34e when the plunger is activated, as described in International Patent Application No. PCT/US2014/026511, filed Mar. 13, 2014 and titled “FLUID DISPENSING CONTROL SYSTEMS AND METHODS,” the entirety of which is incorporated by reference herein. The servo motor 2406 can, in turn, be connected to a fluid dispensing control circuit board 2408 that can receive input from a trap position sensor 2410 and a syringe position sensor 2412. In some embodiments, the controller 2404 can also include a gas burst control circuit board 2412 configured to control the amount of gas supplied to the gas lumens 34a-d of the sheath 100. The gas burst circuit board 2412 can be connected, for example, to a filter 2414, a first solenoid valve 2416, a pressure regulator 2418, a pressure transducer 2420, a flow meter 2422, and a solenoid valve 2424. The solenoid valves 2416, 2424 can act as a reservoir to control the amount of gas available for bursting. The flow meter 2422 can act as a safety back-up to ensure that not too much gas burst is provided to the patient (i.e., so that the patient is not over-pressurized). A display circuit board 2426 can be configured to display information about the fluid dispensing and gas dispensing, such as the level of fill of the syringes, the volume of gas available for burst, and/or the flow rate of the fluid or gas. A tube set gas filter 2428 and a gas burst direction valve 2430 ensure that the burst gas is properly guided to and through the gas lumens 34a-d.

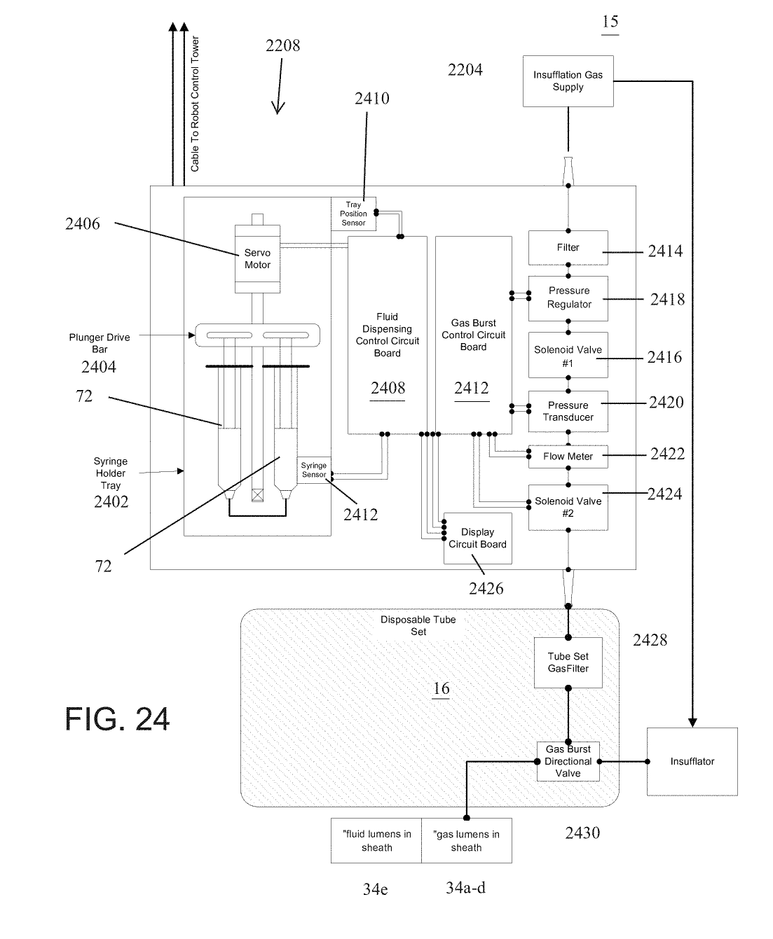

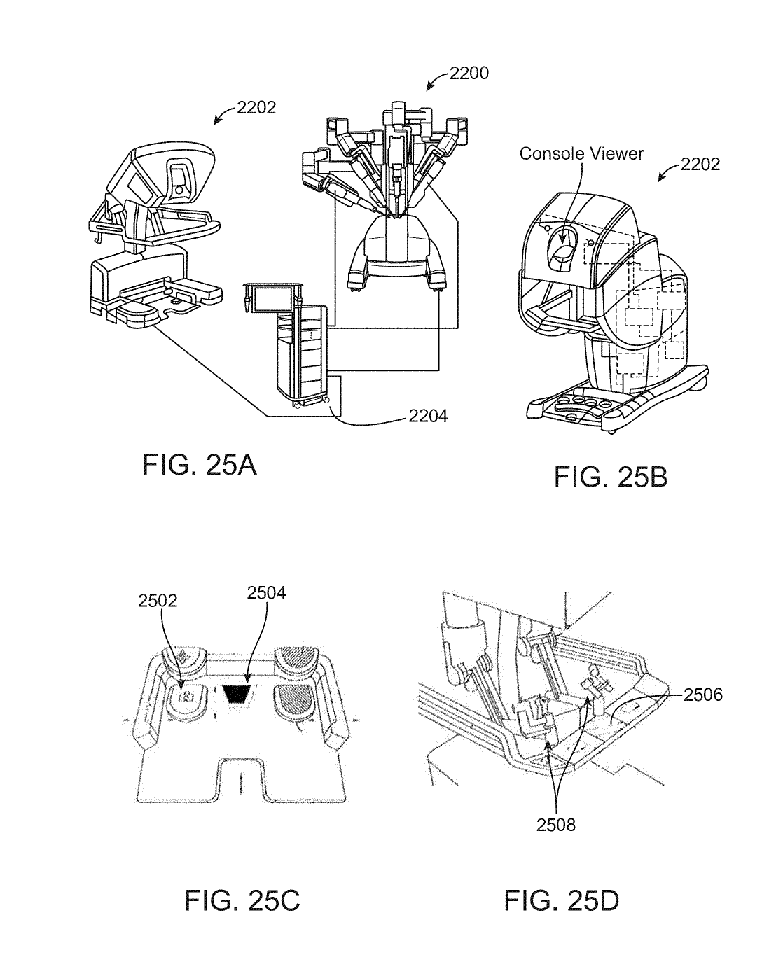

Referring to FIGS. 25A-25D, the controls at the console 2202 can be configured to include user controls for the sheath 100 (and robot 2200) in a variety of different ways. For example, as shown in FIG. 25C, a camera foot switch 2502 can activate the laparoscope while a footswitch 2504 can be used to activate the gas and/or fluid through the sheath 100. In other embodiments, shown in FIG. 25D, a touch pad 2506 or hand controls 2508 can be used to activate the sheath 100 (i.e., supply gas or fluid). For example, in the touch screen control method, using the touch screen control pad 2506, the surgeon can press a “scope clean” button to start one auto-clean cycle of the sheath. In the footswitch control method, the surgeon can start an auto-cycle by pressing the footswitch 2504. In the hand control method, the surgeon can use the hand controls 2508 to start an auto-clean cycle of the sheath, such as by pinching two finger controls simultaneously or spreading finger controls apart. In some embodiments, the same button or control can be used to activate both fluid and burst based upon a staggered initiation of the steps.

In some embodiments, the surgeon can be alerted to the start of a cleaning cycle by an audible beep or a signal displayed on the console viewer.

In some embodiments, the surgeon can activate dispensing of fluid and blow-off of air independently. By selecting this option, dispensing and blow-off cycles can be controlled by the footswitch or the touch screen panel.

Further, in some embodiments, voice control can be used to activate the sheath, i.e., the surgeon can issue an audible command, such as “clean scope,” “start scope clean cycle,” “rinse lens,” or “gas burst” through a console microphone.

Further, in some embodiments, the controller 2204 can include plunger position sensors, such as encoders, to notify a “syringe empty” or “syringe low” alarm.

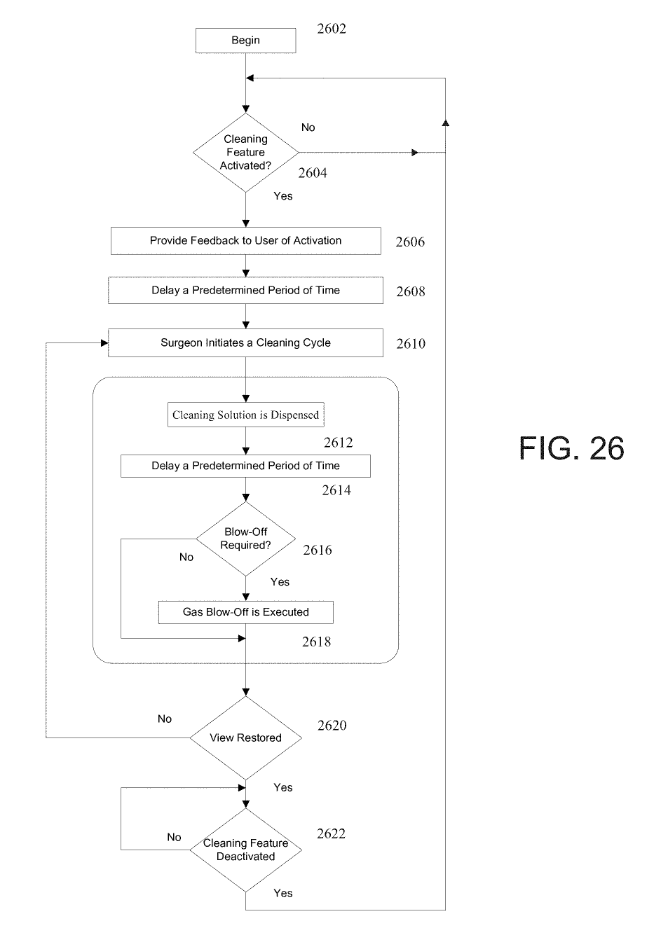

An exemplary flow chart for activating a cleaning feature of a sheath with a robotic surgical system is shown in FIG. 26. At step 2602, the process begins (i.e., gas is continuously provided to the sheath to provide defogging). At step 2604, it can be determined whether the scope cleaning feature (fluid and gas burst) is activated. If so, then the user can be provided feedback of activated at step 2606 and a predetermined delay time can occur at step 2608. If the cleaning feature is not activated, then the step can be repeated until activated. At step 2610, the surgeon can then initiate a cleaning cycle (e.g., by pushing a foot pedal, activating a switch on the control board, etc.). In doing so, cleaning solution can be dispensed at step 2612. A predetermined time period can be activated at step 2614, and then it can be determined at step 2616 whether gas blow-off is required. If so, gas blow-off can be executed at step 2618 and it can be determined whether view can be restored at step 2620 (the gas blow-off can be controlled by the surgeon, can automatically occur after a predetermined period of time, or can occur automatically based upon sensors in the system). If gas blow off is not required, then it can again be determined whether view has been restored at step 2620. If not, then the cleaning cycle can be imitated again at step 1610. If so, then it can be determined whether the cleaning feature should be deactivated at step 1612. The process can then be repeated.

In some embodiments, the cleaning and gas burst features can occur automatically if sensors on the tip of the laparoscope indicate a certain level of debris or if the imaging reaches a certain cloudiness level.

### Case Examples

### Example Case 1

A robotic gastric sleeve procedure was performed with Intuitive Surgical's da Vinci® robotic surgical system at Reading Hospital in Reading, Pa. A sheath configured substantially as shown in FIGS. 1A and 2 was placed over the laparoscope of the surgical system and registered and stabilized using the camera mount configured substantially as shown in FIGS. 5A-5B. A Stryker pnuemosure insufflator was used to flow gas at a pressure of 15 mmHG through the lumens of the sheath to clear the lens of the laparoscope. An average time to defog after starting the airflow through the sheath was reported as 2.5 seconds. A fluid, such as the docusate sodium mix described in the '8014 application, incorporated by reference above, was injected through the sheath two times during the procedure to clear the lens with an average time of 20 seconds to restore view after injection of the fluid. The laparoscope did not have to be removed from the patient for cleaning or defogging throughout the entire gastric sleeve procedure.

### Example Case 2

A robotic gastric sleeve procedure was performed with Intuitive Surgical's da Vinci® robotic surgical system at Reading Hospital in Reading, Pa. A sheath configured substantially as shown in FIGS. 1A and 2 was placed over the laparoscope of the surgical system and registered and stabilized using the camera mount configured substantially as shown in FIGS. 5A-5B. A Stryker pnuemosure insufflator was used to flow gas at a pressure of 15 mmHG through the lumens of the sheath to clear the lens of the laparoscope. An average time to defog starting the airflow through the sheath was reported as 2.5 seconds. A fluid, such as the docusate sodium mix described in the '8014 application, incorporated by reference above, was injected through the sheath three times during the procedure to clear the lens with an average time of 5 seconds to restore view after injection of the fluid. The laparoscope did not have to be removed from the patient for cleaning or defogging throughout the entire gastric sleeve procedure.

### Example Case 3

A robotic gastric sleeve procedure was performed with Intuitive Surgical's da Vinci® robotic surgical system at Reading Hospital in Reading, Pa. A sheath configured substantially as shown in FIGS. 1A and 2 was placed over the laparoscope of the surgical system and registered and stabilized using the camera mount configured substantially as shown in FIGS. 5A-5B. A Stryker pnuemosure insufflator was used to flow gas at a pressure of 15 mmHG through the lumens of the sheath to clear the lens of the laparoscope. An average time to defog starting the airflow through the sheath was reported as 2.5 seconds. A fluid, such as the docusate sodium mix described in the '8014 application, incorporated by reference above, was injected through the sheath 5 times during the procedure to clear the lens with an average time of 8 seconds to restore view after injection of the fluid. The laparoscope did not have to be removed from the patient for cleaning or defogging throughout the entire gastric sleeve procedure.

### Example Case 4

A robotic hysterectomy was performed with Intuitive Surgical's da Vinci® robotic surgical system at Riverside Methodist Hospital in Columbus, Ohio. A sheath configured substantially as shown in FIGS. 1A and 2 was placed over the laparoscope of the surgical system and registered and stabilized using the camera mount configured substantially as shown in FIGS. 5A-5B. A Storz insufflator was used to flow gas through the lumens of the sheath to clear the lens of the laparoscope. The laparoscope did not have to be removed from the patient for cleaning or defogging throughout the entire gastric sleeve procedure.

### Example Case 5

A robotic gastric sleeve procedure was performed with Intuitive Surgical's da Vinci® robotic surgical system at Reading Hospital in Reading, Pa. A sheath configured substantially as shown in FIGS. 1A and 2 was placed over the laparoscope of the surgical system and registered and stabilized using the camera mount configured substantially as shown in FIGS. 5A-5B. A Stryker pnuemosure insufflator was used to flow gas at a pressure of 15 mmHG through the lumens of the sheath to clear the lens of the laparoscope. A fluid, such as the docusate sodium mix described in the '8014 application, incorporated by reference above, was injected through the sheath 3 times during the procedure to clear the lens with an average time of 5 seconds to restore view after injection of the fluid. The laparoscope did not have to be removed from the patient for cleaning or defogging throughout the entire gastric sleeve procedure.

It is to be appreciated that the above described features of adjustment in the component design, alone or in combination of a suitable coupling mount with the other resiliently adjustable scope mount features permits the use of the various sheath embodiments to be utilized in a wide variety of surgical environments.

### CONCLUSION

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A system for clearing a lens of a robotic scope, comprising:
a sheath elongate body having an inner surface and an outer surface and a plurality of lumens extending between the inner surface and the outer surface, the inner surface having an interior dimension selected to receive the robotic scope;
an attachment mechanism configured to connect the sheath to the robotic scope;
a gas connection port configured to provide gas to a first lumen of the plurality of lumens;
a fluid connection portion configured to provide fluid to a second lumen of the plurality of lumens;
a deflector assembly attached to a distal end of the sheath configured to deflect gas and fluid from the plurality of lumens over a lens of the scope; and
an activation element remotely located relative to the sheath and configured to provide gas or fluid to the plurality of lumens when activated such that the gas or fluid flows over the lens.

2. The system of claim 1, wherein the activation element is located on a console, the console including elements configured to control the robotic scope.

3. The system of claim 2, wherein the activation element is a foot pedal of the console.

4. The system of claim 2, wherein the activation element is a hand control of the console.

5. The system of claim 1, wherein the activation element is a voice activated element.

6. The system of claim 1, further comprising a controller, the controller configured to automatically provide gas through the first lumen or fluid through the second lumen based upon a trigger.

7. The system of claim 6, wherein the trigger is a predetermined period of time of operation of the laparoscope.

8. The system of claim 6, wherein the trigger is a predetermined period of time of flowing gas down the first lumen at a first pressure.

9. The system of claim 8, wherein automatically providing gas or fluid comprises providing fluid down the second lumen, and wherein the controller is further configured to automatically provide gas down the first lumen at a second pressure that is higher than the first pressure after automatically providing fluid down the second lumen.

10. The system of claim 6, wherein the system includes one or more sensors, and wherein the trigger is a reading from the one or more sensors.

11. The system of claim 10, wherein the one or more sensors are configured to detect debris over the lens of the scope.

12. The system of claim 6, wherein the trigger is a cloudiness level of an image produced by the laparoscope.

13. A system for clearing a lens of a robotic scope, comprising:
a sheath elongate body having an inner surface and an outer surface and a plurality of lumens extending between the inner surface and the outer surface, the inner surface having an interior dimension selected to receive the robotic scope;
an attachment mechanism configured to connect the sheath to the robotic scope;
a gas connection port configured to provide gas to a first lumen of the plurality of lumens;
a fluid connection portion configured to provide fluid to a second lumen of the plurality of lumens;
a deflector assembly attached to a distal end of the sheath configured to deflect gas or fluid from the plurality of lumens over a lens of the scope; and
a controller, the controller configured to automatically provide gas or fluid through the one or more lumens based upon a trigger.

14. The system of claim 13, wherein the trigger is a predetermined period of time of operation of the laparoscope.

15. The system of claim 13, wherein the trigger is a predetermined period of time of flowing gas down the first lumen at a first pressure.

16. The system of claim 15, wherein automatically providing gas or fluid comprises providing fluid down the second lumen, and wherein the controller is further configured to automatically provide gas down the first lumen at a second pressure that is higher than the first pressure after automatically providing fluid down the second lumen.

17. The system of claim 13, wherein the system includes one or more sensors, and wherein the trigger is a reading from the sensor.

18. The system of claim 17, wherein the one or more sensors are configured to detect debris over the lens of the scope.

19. The system of claim 13, wherein the trigger is a cloudiness level of an image produced by the laparoscope.
